(19) United States
(12) Patent Application Publication
(10) Pub. No.: US 2024/0141359 A1
(43) Pub. Date: May 2, 2024

(54) GENOME EDITING FOR TREATING MUSCULAR DYSTROPHY

(71) Applicant: UNIVERSITY OF MASSACHUSETTS, Boston, MA (US)

(72) Inventors: Charles P. Emerson, JR., Lyndon, VT (US); Scot A. Wolfe, Winchester, MA (US)

(21) Appl. No.: 18/278,338

(22) PCT Filed: Feb. 23, 2022
(86) PCT No.: PCT/US22/17560

§ 371 Date: Aug. 22, 2023

Related U.S. Application Data
(60) Provisional application No. 63/152,669, filed on Feb. 23, 2021.

(51) Int. Cl. C12N 15/113; C12N 9/22; C12N 15/86

(52) U.S. Cl. CPC C12N 15/1138; C12N 9/22; C12N 15/86; C12N 2310/20; C12N 2750/14143

(57) ABSTRACT

The present invention is related to the field of genetic engineering. In particular, the repair, reversion and/or conversion of genetic mutations that are linked to a muscular dystrophy disease. Specifically contemplated are gene editor nuclease proteins or base editor proteins that are targeted to the muscular dystrophy genetic mutations or pathogenic variants. Such gene editor nuclease proteins include, but are not limited to Cas12a nuclease proteins and adenine base editor proteins. Repair, reversion and/or disruption of the genetic mutation or pathogenic variant reduces at least one symptom of a muscular dystrophy disease.

### FIELD OF THE INVENTION

The present invention is related to the field of genetic engineering. In particular, the repair, reversion and/or conversion of genetic mutations that are linked to a muscular dystrophy disease. Specifically contemplated are gene editor nuclease proteins that are targeted to the muscular dystrophy genetic mutations. Such gene editor nuclease proteins include, but are not limited to Cas12a nuclease proteins and adenine base editor proteins. Repair, reversion and/or conversion of the genetic mutation reduces at least one symptom of a muscular dystrophy disease.

### BACKGROUND

There is no cure for muscular dystrophy. Physical therapy, braces, and corrective surgery, however, may help with some symptoms. Assisted ventilation may be required in those with weakness of breathing muscles. Medications used include steroids to slow muscle degeneration, anticonvulsants to control seizures and some muscle activity, and immunosuppressants to delay damage to dying muscle cells. Clinical outcomes depend on the specific type of disorder.

What is needed in the art is a therapy designed to permanently remedy the genetic basis of muscular dystrophy.

### SUMMARY OF THE INVENTION

The present invention is related to the field of genetic engineering. In particular, the repair, reversion and/or inactivation of genetic mutations or pathogenic variants that are linked to a muscular dystrophy disease. Specifically contemplated are gene editor nuclease proteins that are targeted to the muscular dystrophy genetic variations that are associated with disease progression. Such gene editor nuclease proteins include, but are not limited to Cas12a nuclease proteins and cytosine and adenine base editor proteins. Repair, reversion and/or inactivation of the genetic mutation or pathogenic variation reduces at least one symptom of a muscular dystrophy disease.

In one embodiment, the present invention contemplates a method, comprising: a) providing; i) a mammal exhibiting at least one symptom of a muscular dystrophy disease and a genetic mutation; ii) a nuclease, base editor or prime editor protein targeted to said genetic mutation; and b) editing said genetic mutation or pathogenic variant such that at least one symptom is reduced. In one embodiment, the genetic mutation is in a muscle stem cell or muscle tissue. In one embodiment, the pathogenic variant is in a 4qA locus which includes DUX4 disease gene and associated pathological variant sequences known as the A haplotype. In one embodiment, the genetic variant of the A haplotype associated with clinical disease comprises a polyadenylation signal sequence for the DUX4 disease gene. In one embodiment, the A haplotype genetic variant associated with disease is an ATTAAA sequence. In one embodiment, the genetic mutation is within a nucleic acid sequence of a gene including, but not limited to, a DMD gene, a COL6A gene, a DYSF gene, an ANO5 gene, an EMD gene, an LMNA gene, a DUX4 gene, a DYSF gene, a DMPK gene, a ZNF9 (CNBP) gene and/or a PABPN1 gene. In one embodiment, the nuclease is an enhanced AsCas12a (enAsCas12a) nuclease. In one embodiment, the enAsCas12a nuclease binds to a crRNA3 guide RNA. In one embodiment, the nuclease is an adenine base editor. In one embodiment, the adenine base editor is a TadA-8e adenine base editor. In one embodiment, the method further comprises administering the enAsCas12a nuclease into the muscle cell, myoblast progenitor cell and/or satellite (muscle stem) cell. In one embodiment, the method further comprises differentiating the edited myoblast cell into a muscle tissue. In one embodiment, the muscle cell, and/or satellite cell comprises in vivo human muscle tissue. In one embodiment, the muscle and/or myoblast cell is an in vitro human muscle cell line. In one embodiment, the in vitro human muscle cell line is an immortalized facioscapulohumeral muscular dystrophy (FSHD) patient myoblast cell line. In one embodiment, the immortalized FSHD patient myoblast cell line is a 15Abic cell line. In one embodiment, the muscle, myoblast cell and/or satellite cell is an FSHD mouse muscle, myoblast cell and/or satellite cell. In one embodiment, the FSHD mouse muscle cell is an FLExDUX4 mouse muscle, myoblast cell and/or satellite cell. In one embodiment, the muscular dystrophy is facioscapulohumeral muscular dystrophy. In one embodiment, the administering further comprises a nanoparticle comprising the nuclease protein. In one embodiment, the administering further comprises an associated adenovirus comprising the nuclease protein.

In one embodiment, the present invention contemplates a nuclease protein targeted to a muscular dystrophy genetic mutation or pathogenic variant. In one embodiment, the nuclease protein is an enAsCas12a nuclease protein. In one embodiment, the enAsCas12a nuclease binds to a crRNA3 guide RNA. In one embodiment, the nuclease is an adenine base editor. In one embodiment, the adenine base editor is a TadA-8e adenine base editor. In one embodiment, the muscular dystrophy genetic mutation is within a nucleic acid sequence of a gene including, but not limited to, a DMD gene, a COL6A gene, a DYSF gene, an ANO5 gene, an EMD gene, an LMNA gene, a DUX4 gene, a DYSF gene, a DMPK gene, a ZNF9 (CNBP) gene and/or a PABPN1 gene.

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “muscular dystrophy” as used herein, refers to any medical condition diagnosed by the results of muscle biopsy, increased creatine phosphokinase (CpK3), electromyography, genetic testing, physical examination and medical history. Other diagnostic tests include, but are not limited to, chest X-ray, echocardiogram, computed tomography and magnetic resonance imaging.

The term “muscle cell” as used herein, refers to any type of cell associated with the heart muscle, or skeletal muscle for these tissues. Examples include muscle fibers, myotubes, myocytes, myoblasts, satellite cells and cardiomyocytes.

The term “genetic mutation” as used herein, refers to a permanent alteration in the DNA sequence that makes up a gene, such that the sequence differs from a wild type gene. Mutations range in size; they can affect anywhere from a single base pair to a large segment of a chromosome (e.g., repeat expansions).

The term “pathogenic variation” as used herein, refers to a genetic alteration that increases an individual's susceptibility or predisposition to a certain disease or disorder. When such a variant (or mutation) is inherited, development of symptoms is more likely, but not certain. Pathogenic variations range in size; they can affect anywhere from a single base pair to a large segment of a chromosome (e.g., 4qA allele in the DUX4 locus on Chromosome 4; (PMID 20724583)).

The term “polyadenylation sequence” or “polyadenylation signal sequence” (PAS) as used herein, refers to a sequence within the DNA that instructs RNA polymerase II and its associated proteins to cleave the RNA transcript and polyadenylate the 3′ end of the RNA to stabilize the sequence. The polyadenylation signal sequence has a consensus of ATAAAA, but variants of this sequence are also functional, such as ATTAAA of the A haplotype of the 4qA allele of DUX4 (PMID 20724583). Note that other sequences surrounding the PAS may also be critical to recognition by the RNA polymerase complex for polyadenylation. The disruption of the PAS and/or its surrounding sequence can abrogate RNA transcript processing and polyadenylation.

The term “nuclease protein” as used herein, refers to a protein capable of cleaving the phosphodiester bonds between nucleotides of nucleic acids. Nucleases can create either single stranded breaks (e.g., a nickase) and double stranded breaks. Such nuclease proteins include, but are not limited to Cas proteins, base editors or prime editors.

The term “locus” as used herein, refers to a specific physical location of a gene, allele or other DNA sequence on a chromosome.

The term “bind”, “binding”, or “bound” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That may be typical when the binding component may be an enzyme and the analyte may be a substrate for the enzyme. Reactions resulting from contact between the binding agent and the analyte are also within the definition of binding for the purposes of the present invention.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by the same series in reverse and then by 30 or so base pairs known as “spacer DNA”. The spacers are short segments of DNA from a virus and may serve as a ‘memory’ of past exposures to facilitate an adaptive defense against future invasions (PMID 25430774).

As used herein, the term “Cas” or “CRISPR-associated (cas)” refers to genes often associated with CRISPR repeat-spacer arrays (PMID 25430774).

As used herein, the term “Cas9” refers to a nuclease from Type II CRISPR systems, an enzyme specialized for generating double-strand breaks in DNA, with two active cutting sites (the HNH and RuvC domains), one for each strand of the double helix. Jinek combined tracrRNA and crRNA (spacer RNA) into a “single-guide RNA” (sgRNA) molecule that, mixed with Cas9, could find and cleave DNA targets through Watson-Crick pairing between the guide sequence within the sgRNA and the target DNA sequence (PMID 22745249). There have been substantial efforts to broaden the targeting specificity of SpyCas9 through mutations that increase the number of PAMs that can be recognized. Two of the most prominent modified versions of Cas9 are xCas9 (Hu et al. 2018 (PMID 29512652)) and Cas9-NG (Nishimasu et al. 2018 (PMID 30166441)), both of which permit targeting some additional PAM elements.

As used herein, the term “Cas12a” (also know as Cpf1) refers to a nuclease from Type II CRISPR systems, an enzyme specialized for generating double-strand breaks in DNA, with one active cutting site (the RuvC domain), that can cleave both strands of the double helix (PMID 26422227). Cas12a nucleases utilize a single crRNA to find and cleave DNA targets through Watson-Crick pairing between the guide sequence within the crRNA and the target DNA sequence (PMID 26422227; 27096362). There have been substantial efforts to broaden the targeting specificity of Cas12a nucleases through mutations that increase the number of PAMs that can be recognized. One of the most prominent modified versions of Cas12a is enAsCas12a (Kleinstiver et al. 2019 (PMID 30742127)), which permits targeting some additional PAM elements beyond the 5′ TTTV PAM normally recognized by AsCas12a.

As used herein, the term “guide RNA” refers to an RNA that programs a CRISPR-Cas protein to recognize a target site in the genome. This could be a crRNA, crRNA/tracrRNA, sgRNA or a pegRNA depending on the type of Cas9 protein and the modifications that have been made to the protein to incorporate extra functionality.

As used herein, the term “catalytically active Cas9” refers to an unmodified Cas9 nuclease comprising full nuclease activity.

The term “base editor” as used herein, refers to a fusion protein typically containing two components: an adenine or cytidine deaminase and a Cas9/sgRNA complex (e.g., Cas12a/crRNA complex), where the Cas9 component is mutated so that it cannot produce a double-strand break. Typically the Cas9 component will be a strand specific nickase for the adenine base editor (ABE) or cytosine base editor (CBE), although nuclease-dead versions of Cas9 (or Cas12a) can also be used. These systems allow the strand-specific conversion of cytosine to uracil or adenine to guanine within the DNA (Huang, et. al. 2021 (PMID 33462442)). These base editor systems can be used to revert point mutations, introduce stop codons, disrupt splicing sequences or other transcriptional or post-transcriptional regulatory elements, all of which can be valuable for therapeutic applications.

The term “nickase” as used herein, refers to a nuclease that cleaves only a single DNA strand, either due to its natural function or because it has been engineered to cleave only a single DNA strand. Cas9 nickase variants (e.g. nSpCas9, nCas9) that have either the RuvC or the HNH domain mutated provide control over which DNA strand is cleaved and which remains intact (Jinek, et al. 2012 (PMID 22745249) and Cong, et al. 2013 (PMID 23287718)).

The term “cytidine deaminase” refers to a protein domain that converts cytosine to uracil in the target DNA strand. In the context of a cytosine base editor, the cytidine deaminase drives the conversion of a C-G base pair to a T-A base pair. There are a large number of different cytidine deaminases that have been used in cytosine base editors—natural deaminases, such as rAPOBEC1, and engineered variants such as BE4 (Huang, et. al. 2021 (PMID 33462442) and references therein). The type of cytidine deaminase domain can be swapped within cytosine base editors to change the base conversion efficiency in different sequence contexts.

The term “adenine deaminase” refers to a protein domain that converts adenine to inosine in the target DNA strand. In the context of an adenine base editor, the adenine deaminase drives the conversion of an A-T base pair to a G-C base pair. There are a number of different adenine deaminases that have been evolved for use in adenine base editors, such as TadA7.10 and TadA8e (Huang, et. al. 2021 (PMID 33462442) and references therein). The type of adenine deaminase domain can be swapped within adenine base editors to change the base conversion efficiency in different sequence contexts.

The term, “trans-activating crRNA”, “tracrRNA” as used herein, refers to a small trans-encoded RNA. For example, CRISPR/Cas (clustered, regularly interspaced short palindromic repeats/CRISPR-associated proteins) constitutes an RNA-mediated defense system, which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into effector complexes, where the crRNA guides the complex to the invading nucleic acid and the Cas proteins degrade this nucleic acid. There are several pathways of CRISPR activation, one of which requires a tracrRNA, which plays a role in the maturation of crRNA. TracrRNA is complementary to base pairs with a pre-crRNA forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM may comprise a trinucleotide sequence having a single G residue (e.g., a single G PAM), or a trinucleotide sequence having two consecutive G residues (e.g., a dual G PAM). The PAM specificity may be a function of the DNA-binding specificity of the Cas9 protein (e.g., a “protospacer adjacent motif recognition domain” at the C-terminus of Cas9).

As used herein, the term “sgRNA” refers to single guide RNA used in conjunction with CRISPR associated systems (Cas). sgRNAs are a fusion of crRNA and tracrRNA and contain nucleotides of sequence complementary to the desired target site (Jinek, et al. 2012 (PMID 22745249)). Watson-Crick pairing of the sgRNA with the target site permits R-loop formation, which in conjunction with a functional PAM permits DNA cleavage or in the case of nuclease-deficient Cas9 allows binds to the DNA at that locus.

The term “primer binding site” as used herein, refers to a specific nucleic acid sequence within the pegRNA that is complementary to the 3′ end of the nicked DNA strand. This allows annealing of the free 3′ end of the genomic DNA for extension by the reverse transcriptase based on the template sequence encoded in the pegRNA.

The term, “prime editing guide RNA molecule” or “pegRNA molecule” as used herein, refers to a Cas9 guide RNA molecule that encodes the crRNA-tracrRNA fused to a primer binding site (PBS) and a reverse transcriptase template. The primer binding site hybridizes to a desired genomic sequence released by the binding and cleavage of the Cas9 nickase. The 3′ end of the genomic sequence is extended by the reverse transcriptase based on the reverse transcriptase template sequence.

The term “editing” as used herein, refers to a genetic manipulation of a DNA sequence. Such a manipulation includes, but is not limited to, a base conversion, a sequence insertion and/or a sequence deletion.

The term “prime editing” as used herein, is a genome editing technology by which the genome of living organisms may be modified. Prime editing manipulates the genetic information of a targeted DNA site to essentially “rewrite” the coded sequences.

The term “prime editor” or “PE” as used herein, is a fusion protein comprising a catalytically impaired Cas9 endonuclease that can nick DNA fused to an engineered reverse transcriptase enzyme, and a prime editing guide RNA (pegRNA). The pegRNA is capable of programming the nCas9 to recognize a target site with the encoded crRNA-tracrRNA (a as single guide RNA). The resulting nicked genomic DNA can be extended by the reverse transcriptase based on the pegRNA template sequence to contain a new sequence. Once one strand is recoded, cellular DNA repair pathways can cause conversion of the local DNA sequence to match the new sequence. Such manipulation includes, but is not limited to, insertions, deletions, and base-to-base conversions without the need for double strand breaks (DSBs) or donor DNA templates. For example, such prime editing may be performed by a Cas9 CRISPR platform programmed with a pegRNA, such as a catalytically impaired Cas9 nickase platform with an appropriate reverse transcriptase.

The term, “nuclear localization signal sequence” or “NLS”, as used here refers to an amino acid sequence that ‘tags’ a protein for import into the cell nucleus by nuclear transport. Typically, this signal includes one or more short sequences of positively charged lysines or arginines exposed on the protein surface. For example, an NLS includes but is not limited to an SV40 NLS (PKKKRKV), a bipartite SV40 NLS (BP-SV40 NLS; KRTADGSEFESPKKKRKV), a variant bipartite SV40 NLS (vBP-SV40 NLS; KRTADSSHSTPPKTKRKV), a Nucleoplasmin NLS (KRPAATKKAGQAKKKKLD) or a C-myc NLS (PAAKRVKLD).

As used herein, the term “orthogonal” refers targets that are non-overlapping, uncorrelated, or independent. For example, if two orthogonal Cas9 isoforms were utilized, they would employ orthogonal sgRNAs that only program one of the Cas9 isoforms for DNA recognition and cleavage (Esvelt, et al. 2013 (PMID 24076762); Edraki, et. al 2018 (PMID 30581144)). For example, this would allow one Cas9 isoform (e.g. S. pyogenes Cas9 or SpCas9) to function as a nuclease or nickase programmed by a sgRNA that may be specific to it, and another Cas9 isoform (e.g. N. meningitidis Cas9, Nm1Cas9 or Nme2Cas9) to operate as a nuclease dead Cas9 that provides DNA targeting to a binding site through its PAM specificity and orthogonal sgRNA. Other Cas9s include S. aureus Cas9 or SaCas9 and A. naeslundii Cas9 or AnCas9.

The term “base pairs” as used herein, refer to specific nucleobases (also termed nitrogenous bases), that are the building blocks of nucleotide sequences that form a primary structure of both DNA and RNA. Double stranded DNA may be characterized by specific hydrogen bonding patterns, base pairs may include, but are not limited to, guanine-cytosine and adenine-thymine) base pairs.

The term “specific genomic target” as used herein, refers to any pre-determined nucleotide sequence capable of binding to a Cas9 protein contemplated herein. The target may include, but may be not limited to, a nucleotide sequence complementary to a programmable DNA binding domain or an orthogonal Cas9 protein programmed with its own guide RNA, a nucleotide sequence complementary to a single guide RNA, a protospacer adjacent motif recognition domain, an on-target binding sequence and an off-target binding sequence.

The term “on-target binding sequence” as used herein, refers to a subsequence of a specific genomic target that may be completely complementary to a programmable DNA binding domain and/or a single guide RNA sequence.

The term “off-target binding sequence” as used herein, refers to a subsequence of a specific genomic target that may be partially complementary to a programmable DNA binding domain and/or a single guide RNA sequence.

The term “bystander editing” or “bystander effect” as used herein refers to the conversion by an ABE or CBE of a nearby base pair that is not the target position where editing is desired (Huang, et. al. 2021 (PMID 33462442)). Such a bystander edit can result in an undesired mutation to a gene or a regulatory element that may alter the function of the gene or regulatory element in an undesired manner.

The term “fails to bind” as used herein, refers to any nucleotide-nucleotide interaction or a nucleotide-amino acid interaction that exhibits partial complementarity, but has insufficient complementarity for recognition to trigger the cleavage of the target site by the Cas9 nuclease. Such binding failure may result in weak or partial binding of two molecules such that an expected biological function (e.g., nuclease activity) fails.

The term “cleavage” as used herein, may be defined as the generation of a break in the DNA. This could be either a single-stranded break or a double-stranded break depending on the type of nuclease that may be employed.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target, the specific inclusion of new sequence through the use of an exogenously supplied DNA template, or the conversion of one DNA base to another DNA base. Such a specific genomic target includes, but may be not limited to, a chromosomal region, mitochondrial DNA, a gene, a promoter, an open reading frame or any nucleic acid sequence.

The term “delete”, “deleted”, “deleting” or “deletion” as used herein, may be defined as a change in either nucleotide or amino acid sequence in which one or more nucleotides or amino acid residues, respectively, are, or become, absent.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” may be complementary to the sequence “A-C-T-G.” Complementarity can be “partial” or “total.” “Partial” complementarity may be where one or more nucleic acid bases may be not matched according to the base pairing rules. “Total” or “complete” complementarity between nucleic acids may be where each and every nucleic acid base may be matched with another base under the base pairing rules. The degree of complementarity between nucleic acid strands has significant effects on the efficiency and strength of hybridization between nucleic acid strands. This may be of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which may be partially complementary, i.e., “substantially homologous,” to a nucleic acid sequence may be one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This may be not to say that conditions of low stringency are such that non-specific binding may be permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be detected in a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which may be a “homolog” may be defined herein as an oligonucleotide sequence which exhibits greater than or equal to 50% identity to a sequence, when sequences having a length of 100 bp or larger are compared.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “gene of interest” as used herein, refers to any pre-determined gene for which deletion may be desired.

The term “allele” as used herein, refers to any one of a number of alternative forms of the same gene or same genetic locus.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and may be, in a preferred embodiment, free of other genomic nucleic acid).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

As used herein, the term “hybridization” may be used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) may be impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C o t or R o t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

DNA molecules are said to have “5′ ends” and “3′ ends” because mononucleotides are reacted to make oligonucleotides in a manner such that the 5′ phosphate of one mononucleotide pentose ring may be attached to the 3′ oxygen of its neighbor in one direction via a phosphodiester linkage. Therefore, an end of an oligonucleotide may be referred to as the “5′ end” if its 5′ phosphate may be not linked to the 3′ oxygen of a mononucleotide pentose ring. An end of an oligonucleotide may be referred to as the “3′ end” if its 3′ oxygen may be not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “associated with” as used herein, refers to an art-accepted causal relationship between a genetic mutation and a medical condition or disease. For example, it is art-accepted that a patient having an HTT gene comprising a tandem CAG repeat expansion mutation has, or is a risk for, Huntington's disease.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a sample, a compound or a sequence. In one respect, a sample, a compound or a sequence may be derived from an organism or particular species. In another respect, a sample, a compound or sequence may be derived from a larger complex or sequence.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1A presents exemplary genome editing sites in a Dux4 4qA locus.
Top Sequence: A PAS ATTAAA sequence (yellow) was targeted using three different enAsCas12a crRNAs. Yellow arrows indicate the crRNA1 and crRNA2 (5′→3′). The green arrow indicates a crRNA3 target site on the complementary strand. Colored boxes indicate the PAM sequence recognized by enAsCas12a in each respective target site.
Bottom Sequence: A PAS ATTAAA sequence (yellow) within the 4qA allele of DUX4 was targeted by sgRNA1 (blue arrow) with an spCas9/TadA-8e ABE nuclease. Blue box indicates that PAM sequence recognized by the ABE using sgRNA1.

FIG. 1B illustrates the therapeutic strategy of treating muscular dystrophy by gene editing of the DUX4 polyadenylated signal sequence.

FIG. 2 presents exemplary deep sequencing data for the results of enAsCas12a gene editing of the PAS sequence in the 4qA locus in the 15Abic cell line.

FIG. 3 presents exemplary deep sequencing data for the results of TadA-8e ABE gene editing of the PAS sequence in the 4qA locus in the 15Abic cell line.

FIG. 4 presents exemplary data comparing gene editing activity of Abe50, Abe70 and a Cas12a nuclease in cultured primary myoblasts. Estimated InDel rates were determined using ICE editing analysis of Sanger sequencing. The guide target sequences are preliminary.

FIG. 5 presents exemplary data showing identification of edited bases in cultured primary myoblasts using an Abe50 nuclease. A Sanger sequencing trace of DNA amplicon at the 4qA locus of Dux4 from unedited 17abic cells (control, top) and adenine base editor edited cells (50 pmol, bottom). PAS Sequence is highlighted in red box. Site of A to G conversion are shown by red arrows.

FIG. 6 presents exemplary data showing identification of edited bases in cultured primary myoblasts using an Abe70 nuclease. A Sanger sequencing trace of DNA amplicon at the 4qA locus of Dux4 from unedited 17abic cells (control, top) and adenine base-edited cells (70 pmol, bottom)). PAS Sequence is highlighted in red box. Site of A to G conversion are shown by red arrows.

FIG. 7 presents exemplary data showing identification of edited bases in cultured primary myoblasts using a Cas12a nuclease. A Sanger sequencing traces of DNA amplicons at the 4qA locus of Dux4 from unedited 17abic cells (control, top) and enAsCas12a edited cells (80 pmol, bottom) PAS Sequence is highlighted in the red box.

FIG. 8 presents exemplary data showing on/off target data comparing an adenosine base editor with a Cas12a nuclease. The on-target editing rates at the 4qA Dux4 locus for various different treatment groups was assessed by Illumina deep sequencing of amplicons spanning the polyadenylation site (PAS). The off-target rates were estimated from a small number of sequences that map to the 10qA that were amplified by PCR and mapped to the 10qA locus.

FIG. 9 presents exemplary data showing the specificity of on-target gene editing for both adenosine base editors and Cas12a nuclease at the polyadenylation signal sequence. The on-target editing rates at the 4qA Dux4 locus for various different treatment groups was assessed by Illumina deep sequencing of amplicons spanning the polyadenylation site (PAS). Other alleles represent sequences that are edited but that do not disrupt the core ATTAAA PAS sequence.

FIG. 10 presents exemplary data showing a deep sequencing negative control analysis. CRISPResso analysis of Illumina deep sequencing data for the control unedited 15abic sample for PCR amplicons spanning the 4qA Dux4 locus (ON). Off-target (OT) sequences represent amplicons that map to the 10qA locus that represent an off-target site for editing. The red box indicates the PAS sequence element.

FIG. 11 present exemplary data showing a Cas12a deep sequencing analysis.
FIG. 11A: An on-target gene editing CRISPResso analysis of Illumina deep sequencing data for the 15abic sample edited with enAsCas12a for PCR amplicons spanning the 4qA Dux4 locus (ON). The red box indicates the PAS sequence element.
FIG. 11B: An off-target gene editing CRISPResso analysis of Illumina deep sequencing data for the 15abic sample edited with enAsCas12a for PCR amplicons mapped to the 10qA locus off-target site (OT). The red box indicates the PAS sequence element.

FIG. 12 presents exemplary data showing an Abe50 deep sequencing analysis.
FIG. 12A: An on-target gene editing CRISPResso analysis of Illumina deep sequencing data for the 15abic sample edited with 50 pmol adenine base editor (ABE) for PCR amplicons spanning the 4qA Dux4 locus (ON). The red box indicates the PAS sequence element. Arrows shows sites of A to G conversion within the PAS sequence. ABE formulated in 5 μl of Buffer R.
FIG. 12B: An off-target gene editing CRISPResso analysis of Illumina deep sequencing data for the 15abic sample edited with 50 pmol adenine base editor (ABE) for PCR amplicons mapped to the 10qA DUX4 locus off-target site (OT). The red box indicates the PAS sequence element. Arrows shows sites of A to G conversion within the PAS sequence. ABE was formulated in 5 μl of Buffer R.

FIG. 13 presents exemplary data showing a Abe50L deep sequencing analysis.
FIG. 13A: An on-target gene editing CRISPResso analysis of Illumina deep sequencing data for the 15abic sample edited with 50 pmol adenine base editor (ABE) in R buffer for PCR amplicons spanning the 4qA Dux4 locus (ON). The red box indicates the PAS sequence element. Arrows shows sites of A to G conversion within the DUX4 PAS sequence. ABE was formulated in 12 μl of Buffer R.
FIG. 13B: An off-target gene editing CRISPResso analysis of Illumina deep sequencing data for the 15abic sample edited with 50 pmol adenine base editor (ABE) in R buffer for PCR amplicons mapped to the 10qA DUX4 locus off-target site (OT). The red box indicates the PAS sequence element. Arrows shows sites of A to G conversion within the PAS sequence. ABE was formulated in 12 μl of Buffer R.

FIG. 14 presents exemplary data showing a Abe80 deep sequencing analysis.
FIG. 14A: An on-target gene editing CRISPResso analysis of Illumina deep sequencing data for the 15abic sample edited with 80 pmol adenine base editor (ABE) for PCR amplicons spanning the 4qA DUX4 locus (ON). The red box indicates the PAS sequence element. Arrows shows sites of A to G conversion within the PAS sequence.
FIG. 14B: An off-target gene editing CRISPResso analysis of Illumina deep sequencing data for the 15abic sample edited with 80 pmol adenine base editor (ABE) for PCR amplicons mapped to the 10qA DUX4 locus off-target site (OT). The red box indicates the PAS sequence element. Arrows shows sites of A to G conversion within the DUX4 PAS sequence.

FIG. 15 shows an illustration of a clonal analysis in which to measure nucleic acid expression from differentiated myoblasts by RT-qPCR and nanostring techniques in accordance with Example 3.

FIG. 16 presents exemplary data showing that edited PAS sequences modulate DUX4 gene expression as measured by the expression of downstream marker genes. The analysis of DUX4 biomarker RNA expression was performed by Nanostring and RT-qPCR for 15Vbic (15V, healthy control), 15Abic (15A, FSHD affected), Clone 3 (C3) and Clone 8 (C8). PAS edited Clones 3 & 8 have lower Dux4 marker gene expression than the parent unedited 15Abic line.

### DETAILED DESCRIPTION OF THE INVENTION

The present invention is related to the field of genetic engineering. In particular, the repair, reversion and/or inactivation of genetic mutations or pathogenic variants that are linked to a muscular dystrophy disease. Specifically contemplated are gene editor nuclease proteins that are targeted to the muscular dystrophy genetic mutations or pathogenic variants. Such gene editor nuclease proteins include, but are not limited to Cas12a nuclease proteins and adenine base editor proteins. Other types of nucleases (Cas9, CasX), base editors and prime editors programmed with an appropriate crRNA, sgRNA or pegRNA could be utilized. Repair, reversion and/or conversion of the genetic mutation or pathogenic variant reduces at least one symptom of a muscular dystrophy disease.

### I. Muscular Dystrophy

### A. Overview

Muscular dystrophy (MD) has been reported to be a group of muscle diseases that results in increasing weakening and breakdown of skeletal muscles over time. The disorders differ in which muscles are primarily affected, the degree of weakness, how fast they worsen, and when symptoms begin. Many people will eventually become unable to walk, while some types are also associated with problems in other organs. Muscular dystrophy has been linked to as many as thirty (30) different genetic disorders that are usually classified into categories. The most common type is Duchenne muscular dystrophy (DMD), which typically affects males beginning around the age of four. Other types include Becker muscular dystrophy, facioscapulohumeral muscular dystrophy (FSHD), limb-girdle muscular dystrophy, and myotonic dystrophy. These disorders are believed to be caused by mutations in genes that are involved in making muscle proteins. These mutations can either be inherited or de novo and may be X-linked recessive, autosomal recessive, or autosomal dominant. “NINDS Muscular Dystrophy Information Page” National Institute of Neurological Disorders and Stroke, ninds.nih.gov/disorders/md/md (2016); and “Muscular Dystrophy: Hope Through Research”. National Institute of Neurological Disorders and Stroke, ninds.nih.gov/disorders/md/detail_md (2016). See, Table 1.

TABLE 1
Exemplary Types Of Muscular Dystrophy

| Disorder name | Genetic Mutations | Encoded Protein | Description |
|---|---|---|---|
| Becker muscular | DMD | Dystrophin | Becker muscular |
| dystrophy | X chromosome | A protein that is | dystrophy (BMD) is |
| | deletion | part of a complex | a less severe variant |
| | duplication missense | structure | of Duchenne |
| | | involving several | muscular dystrophy |
| | | other protein | and is caused by the |
| | | components. The | production of a |
| | | “dystrophin- | truncated, but |
| | | glycoprotein | partially functional |
| | | complex” helps | form of dystrophin. |
| | | anchor the | Survival is usually |
| | | structural | into old age and |
| | | skeleton | usually affects only |
| | | (cytoskeleton) | boys. |
| | | within the muscle | |
| | | cells, through the | |
| | | sarcolemma of | |
| | | each cell, to the | |
| | | extracellular | |
| | | matrix. | |
| Congenital muscular | COL6A | Collagen VI | General muscle |
| dystrophy | nonsense p.Gln889* | A component of | weakness and |
| | p.Pro260_Lys26linsProPro | the extracellular | possible joint |
| | small insertion. | matrix which | deformities, disease |
| | | forms a | progresses slowly, |
| | | microfibrillar | and lifespan is |
| | | network that is | shortened. Muscle |
| | | found in close | degeneration may be |
| | | association with | mild or severe. May |
| | | the cell and | show severe brain |
| | | surrounding | malformations, such |
| | | basement | as lissencephaly and |
| | | membrane. | hydrocephalus. |
| | | Disorders result | |
| | | in a combined | |
| | | muscle and | |
| | | connective tissue | |
| | | involvement, | |
| | | including | |
| | | weakness, joint | |
| | | laxity and | |
| | | contractures, and | |
| | | abnormal skin | |
| | | findings. | |
| Duchenne muscular | DMD | Dystrophin | Generally affects |
| dystrophy | X chromosome | A protein that is | only diagnosed by |
| | nonsense frameshift | part of a complex | age-related |
| | | structure | difficulty in |
| | | involving several | walking. Lifespans |
| | | other protein | range from 15 to 45. |
| | | components. The | Due to defects in |
| | | “dystrophin- | this assembly, |
| | | glycoprotein | contraction of the |
| | | complex” helps | muscle leads to |
| | | anchor the | disruption of the |
| | | structural | outer membrane of |
| | | skeleton | the muscle cells and |
| | | (cytoskeleton) | eventual weakening |
| | | within the muscle | and wasting of the |
| | | cells, through the | muscle. |
| | | sarcolemma of | |
| | | each cell, to the | |
| | | extracellular | |
| | | matrix. | |
| Distal muscular | DYSF | Dysferlin | Age at onset is |
| dystrophy | human chromosome 2p13 | Also known as | about 20 to 60 |
| | Pro791Arg | dystrophy- | years; symptoms |
| | C1939G | associated fer-1- | include weakness |
| | G3370T 3746delG | like protein. | and wasting of |
| | 4870delT | Anoctamin 5 | muscles of the |
| | ANO5 | An intracellular | hands, forearms, and |
| | | calcium-activated | lower legs; progress |
| | | chloride channel. | is slow and not life- |
| | | Defective plasma | threatening. |
| | | membrane repair | Miyoshi myopathy |
| | | by loss of | causes initial |
| | | annexin | weakness in the calf |
| | | trafficking. | muscles. |
| Emery-Dreifuss | EMD | Emerin | Present in childhood |
| muscular dystrophy | X-linked recessive | Emerin, together | and the early |
| | autosomal dominant | with LEMD3, is | teenaged years with |
| | autosomal recessive | a LEM domain- | contractures. |
| | p.Asp6Glyfs*27 | containing | Clinical signs |
| | LMNA | integral protein | include muscle |
| | R377H | of the inner | weakness and |
| | | nuclear | wasting, stating in |
| | | membrane in | the distal limb |
| | | vertebrates. | muscles and |
| | | Emerin is highly | progressing to |
| | | expressed in | involve the limb- |
| | | cardiac and | girdle muscles. |
| | | skeletal muscle. | Most patients also |
| | | In cardiac | suffer from cardiac |
| | | muscle, emerin | conduction defects |
| | | localizes to | and arrhythmias and |
| | | adherens | dilated |
| | | junctions. | cardiomyopathy. |
| | | Lamin A/C | |
| | | Belongs to the | |
| | | family of laminin | |
| | | nuclear proteins. | |
| Facioscapulohumeral | DUX4 misexpression | Double | Progressive |
| muscular dystrophy | autosomal dominant when | Homeobox 4 | weakness, initially |
| | associated with the collapse | A transcriptional | in the muscles of the |
| | of the D4Z4 repeats to 10 or | activator of | face, shoulders, and |
| | less repeats containing DUX4 | paired-like | upper arms. |
| | disease gene as the terminal | homeodomain | Additional muscles |
| | D4Z4 repeat on chromosome | transcription | are often affected. |
| | 4 (FSHD 1). Recessive when | factor 1 (PITX1). | Symptoms usually |
| | associated with associated | | manifest in |
| | with mutations in SMCHD1 | | adolescence, but |
| | or DNMT3B (FSHD 2) | | clinical disease can |
| | Both FSHD1 and FSHD2 | | also manifest in |
| | require a specific permissive | | early childhood or |
| | haplotype (pathogenic | | adults. Affected |
| | variant) of chromosome 4 | | individuals can |
| | (4qA; 4qA-L) that contains a | | become severely |
| | polymorphism that creates a | | disabled, with 20% |
| | polyadenylation signal | | requiring a wheel |
| | (ATTAAA) distal to the | | chair by age 50. |
| | D4Z4 array | | Penetrance and |
| | 30% de novo mutations | | severity seem to be |
| | | | lower in females |
| | | | compared to males. |
| | | | The cause is de- |
| | | | repression of |
| | | | DUX4, which |
| | | | requires two |
| | | | different types |
| | | | mutations/genetic |
| | | | variants: one |
| | | | mutation causing |
| | | | demethylation of the |
| | | | DUX4 region either |
| | | | through collapse of |
| | | | the D4Z4 repeats or |
| | | | loss of function |
| | | | mutations of |
| | | | proteins (SMCHD1 |
| | | | or DNMT3B) |
| | | | required for proper |
| | | | repressive |
| | | | methylation of the |
| | | | locus, allowing |
| | | | DUX4 transcription, |
| | | | associated with |
| | | | another pathologic |
| | | | sequence variant |
| | | | (e.g., 4qA) |
| | | | containing a |
| | | | polyadenylation |
| | | | signal sequence |
| | | | downstream of |
| | | | DUX4, providing |
| | | | stability to DUX4 |
| | | | messenger RNA and |
| | | | increased likelihood |
| | | | of its translation. |
| Limb-girdle | DYSF | Dysferlin | Affects both boys |
| muscular dystrophy | human chromosome 2p13 | Also known as | and girls. Muscle |
| | autosomal recessive | dystrophy- | weakness, affecting |
| | autosomal dominant | associated fer-1- | both upper arms and |
| | Pro791Arg | like protein. | legs. The recessive |
| | ANO5 | Anoctamin 5 | LGMDs are more |
| | | An intracellular | frequent than the |
| | | calcium-activated | dominant forms, and |
| | | chloride channel. | usually have |
| | | | childhood or |
| | | | teenaged onset. The |
| | | | dominant LGMDs |
| | | | usually show adult |
| | | | onset. Some of the |
| | | | recessive forms |
| | | | have been |
| | | | associated with |
| | | | defects in proteins |
| | | | that make up the |
| | | | dystrophin- |
| | | | glycoprotein |
| | | | complex. Though a |
| | | | person normally |
| | | | leads a normal life |
| | | | with some |
| | | | assistance, in some |
| | | | extreme cases, death |
| | | | from LGMD occurs |
| | | | due to |
| | | | cardiopulmonary |
| | | | complications. |
| Myotonic muscular | DMPK | Dystrophia | Presents with |
| dystrophy | autosomal dominant | myotonica | myotonia (delayed |
| | CTG repeat expansion | protein kinase | relaxation of |
| | ZNF9 (also known as CNBP) | (DMPK) | muscles), as well as |
| | CCTG repeat expansion in | A Ser/Thr protein | muscle wasting and |
| | intron 1 | kinase | weakness. Varies in |
| | | homologous to | severity and |
| | | the MRCK p21- | manifestations and |
| | | activated kinases | affects many body |
| | | and the Rho | systems in addition |
| | | family of | to skeletal muscles, |
| | | kinases. [8] | including the heart, |
| | | ZNF9/CNBP | endocrine organs, |
| | | A protein | and eyes. |
| | | containing seven | |
| | | (7) zinc finger | |
| | | domains and is | |
| | | believed to | |
| | | function as an | |
| | | RNA-binding | |
| | | protein. | |
| | | Also known as, | |
| | | cellular nucleic | |
| | | acid-binding | |
| | | protein (CNBP). | |
| Oculopharyngeal | PABPN1 | Polyadenylate- | Age at onset is 40 to |
| muscular dystrophy | Encoding poly-A short | binding protein 2 | 70 years; symptoms |
| | tandem repeats for GCN[11] | (PABP-2) (also | affect muscles of |
| | to GCN[17]] | known as | eyelids, face, and |
| | | polyadenylate- | throat followed by |
| | | binding nuclear | pelvic and shoulder |
| | | protein | muscle weakness. |
| | | 1(PABPN1)) | |
| | | A protein that is a | |
| | | member of the | |
| | | poly(A)-binding | |
| | | protein family | |
| | | that regulate the | |
| | | translation of | |
| | | some genes into | |
| | | functional | |
| | | proteins. | |

The signs and symptoms of muscular dystrophy include, but are not limited to, progressive muscular wasting, poor balance, scoliosis (i.e., curvature of the spine and the back), progressive inability to walk, waddling gait, calf deformation, limited range of movement, respiratory difficulty, cardiomyopathy, muscle spasms and/or gowers' sign.

Dystrophin protein is found in muscle fiber membrane; its helical nature allows it to act like a spring or shock absorber. Dystrophin links actin in the cytoskeleton and dystroglycans of the muscle cell plasma membrane, known as the sarcolemma. In addition to mechanical stabilization, dystrophin also regulates calcium levels. “DMD gene” Genetics Home Reference medlineplus.gov/genetics/gene/dmd/ (2016); and Lapidos et al., “The Dystrophin Glycoprotein Complex” Circulation Research 94(8): 1023-1031 (2004).

The gene for dystrophin is located on the X chromosome. In males, the lone X chromosome has only one dystrophin gene. If there's a mutation in that gene, a male's muscles will lack dystrophin and slowly degenerate; mutations in the gene for dystrophin were identified as the cause of DMD. A female almost always has two dystrophin genes, one on each X chromosome. Even if one gene is mutated, the other gene suffices to keep dystrophin levels high enough to preserve muscle function in both the heart and skeletal muscles. Nevertheless, it is believed that a small minority of females having both a wild type and a mutated dystrophin gene can still exhibit DMA symptoms.

### B. Facioscapulohumeral Muscular Dystrophy

Facioscapulohumeral muscular dystrophy (FSHD) is a type of muscular dystrophy that preferentially weakens the skeletal muscles of the face, those that position the scapula, and those in the upper arm, overlying the humerus bone (humeral). Weakness of the scapular muscles causes an abnormally positioned scapula. Other areas of the body usually develop weakness as well, such as the abdomen and lower leg, causing foot drop. The two sides of the body are often affected unequally. Symptoms typically begin in early childhood and become noticeable in the teenage years, with 95% of affected individuals manifesting disease by age 20 years.

FSHD is caused by complex genetic changes involving the D4Z4 repeat locus near the telomere on Chromosome 4. The FSHD disease gene DUX4, is encoded by terminal D4Z4 repeat sequences. Typically, DUX4 is expressed during the early development in the male germ line, but not post-natally in somatic tissues. In FSHD, DUX4 is inadequately repressed within muscle tissue. For the majority of individuals (e.g., 95% of FSHD cases), aberrant DUX4 expression is the result of a reduction in the number of D4Z4 macrosatellite repeats (e.g, ˜less than 10 repeats) and results in D4Z4 contraction (FSHD type 1: FSHD1). D4Z4 contraction leads to hypomethylation of the contracted D4Z4 locus and the DUX4 gene. Another 5% of FSHD cases are associated with mutations in chromatin modifying genes (e.g. SMCHD1 or DNMT3B) that disrupt the hypomethylation of the D4Z4 locus also relieving the epigenetic repression of DUX4 (FSHD type 2: FSHD2).

Regardless of the type of genetic driver mutation, disease can only result if the individual has a DUX4 allele associated with the 4qA haplotype which is a common variant (e.g., approximately 50% of alleles in population) at the 3′ end of the DUX4 locus. The 4qA haplotype encodes a functional polyadenylation signal sequence (PAS) that permits a stable accumulation of aberrantly expressed DUX4 transcripts mis-expressed in FSHD muscle, whereas other non-permissive, common haplotypes, such as 4qB, do not function as PAS sites and are not associated with disease progression. Notably, DUX4 expressed in normal germline development utilizes a distal PAS site that is not 4qA sequence dependent. FSHD1 follows an autosomal dominant inheritance pattern, meaning each child of an affected individual has a 50% chance of also being affected.

DUX4 is a sequence-specific transcription factor that regulates a large number of germ line genes that are mis-expressed in FSHD muscle in patients and in differentiated myotube cell cultures derived from muscles of FSHD patients. Mis-expression of DUX4 in myotubes in vitro and muscle fibers in vivo causes muscle damage through cell death and the production of local inflammation, although the exact mechanism of muscle destruction remains poorly defined.

There is no known cure for FSHD, and no pharmaceuticals have proven effective for altering the disease course. Symptoms can be addressed with physical therapy, bracing, and reconstructive surgery. Surgical fixation of the scapula to the thorax is effective in reducing shoulder symptoms in select cases. FSHD is the third most common genetic disease of skeletal muscle affecting 1 in 8,333 to 1 in 15,000 people. Prognosis is extremely variable, with some being severely disabled by age 10 and others never facing significant limitations, although up to 20% of all affected individuals require use of a wheelchair or mobility scooter during adolescence. Life expectancy is highly variable, dependent on disease onset, quality of care and respiratory sufficiency.

Because a 4qA allele comprising a PAS facilitates disease progression of FSHD, genome editing approaches that disrupt this sequence can be expected to treat this disorder. However, as of the present invention, genome editing has been unsuccessful to disrupt the PAS site and prevent DUX4 expression in myotubes from FSHD patients. Jones et al., “Silencing Of DUX4 25 By Recombinant Gene Editing Complexes” US 2020/0017842; and Das et al., “Genome Editing of the Disease Locus D4Z4 as a Means to Ameliorate Gene Misregulation in Facioscapulohumeral Muscular Dystrophy” Thesis, DigiNole: FSU's Digital Repository, fsu.digital.flvc.org/islandora/object/fsu%3A657913, Florida State University.(2018). These studies indicate that deletion of the PAS sequence by Cas9 deletion can reduce DUX4 expression. However, a recent study of PAS modification using TALENs suggests that elimination of the PAS sequence may not prevent DUX4 expression. Joubert et al., “Gene Editing Targeting the DUX4 Polyadenylation Signal: A Therapy for FSHD?” J. Personalized. Med. 11(1):7 (2021); dx.doi.org/10.3390/jpm11010007. Thus it is unclear whether disruption of the PAS sequence by nucleases or base editors or prime editors can reduce DUX4 expression to therapeutic levels.

### II. Cas12a Gene Editing to Treat Muscle Dystrophy

In one embodiment, the present invention contemplates a Cas12a nuclease, a TadA-8e adenine base editor (ABE) or a prime editor that targets an ATTAAA sequence and inactivates the 4qA PAS sequence. In one embodiment, the inactivation is a deletion of one or more nucleotides. In one embodiment, the inactivation is A→G base conversion within the PAS sequence (on either strand). In one embodiment, the Cas12a nuclease, ABE or prime editor is encapsulated within a nanoparticle. In one embodiment, the Cas12a nuclease, ABE or prime editor is encapsulated within an associated-adenovirus. In one embodiment, the Cas12a nuclease, ABE or prime editor is delivered as a protein—RNA complex with additional protein domains or nucleic acid chemical conjugates that facilitate cellular uptake.

Although it is not necessary to understand the mechanism of an invention, it is believed that 4qA PAS targeting does not disrupt DUX4 transcripts that are produced during normal germ line development. It is believed that a distal PAS site remains unaffected by the proposed editing strategy proposed herein thereby minimizing developmental impacts. Nonetheless, all genome editing is anticipated to take place in somatic tissue.

Prior nuclease-based efforts to inactivate the DUX4 4qA PAS sequence have focused on TALENs or Cas9 nucleases. TALEN proteins require heterodimerization to function and although useful for basic research have been more difficult to translate into therapeutics given their large size (each monomer≥2 kb). SpCas9 nuclease does not have a convenient NGG PAM nearby to place the cleavage site within the PAS sequence for disruption. Thus two different sgRNAs need to be used to program SpCas9 nuclease to generate breaks flanking the PAS sequence to delete this element, which will reduce the efficiency of PAS inactivation.

### A. Gene Editing of a 4qA PAS Sequence

Two different approaches have been examined to disrupt a 4qA PAS sequence using: 1) a Cas12a nuclease; or 2) a Cas9 adenine base editor. Immortalized FSHD patient myoblasts (15Abic line) were delivered protein-guide RNA complexes by electroporation. The Cas12a nuclease, was a modified version of AsCas12a. Kleinstiver, et al. Nat Biotechnol 37:276-282 (2019). This nuclease has been modified to increased nuclease activity due by incorporating improved nuclear localization signal (NLS) sequences (e.g., 3xNLS-enAsCas12a).

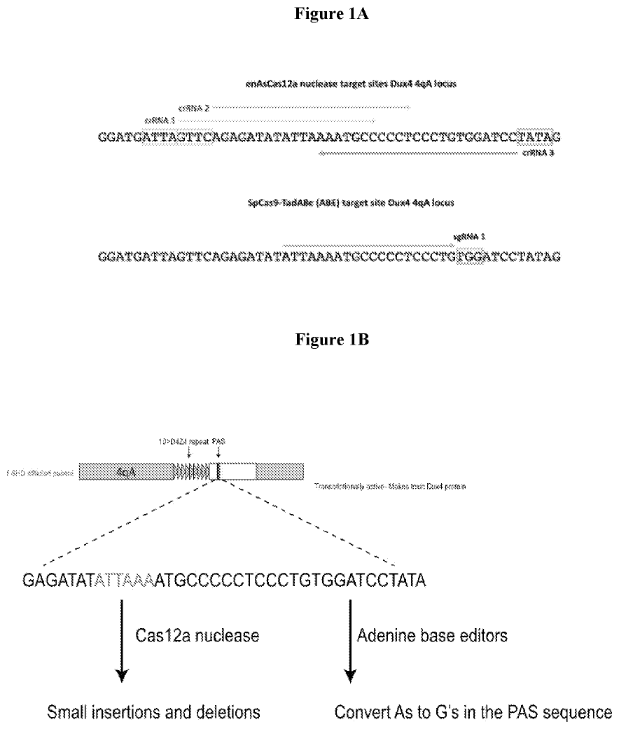

3xNLS-enAsCas12a nuclease was tested at three (3) different target sites that overlap a 4qA PAS sequence. A 3xNLS-enAsCas12a nuclease comprising a crRNA3 sequence produced the most efficient editing at the PAS ATTAAA sequence in a B-LCL cell line that contains an 4qA allele as well as a 15Abic FSHD patient myoblast line. Testing of a Cas9 adenine base editor utilized a modified version of a SpCas9-TadA8e adenine base editor (TadA-8e ABE). Richter et al., Nat Biotechnol 38:883-891 (2020). The TadA-8e ABE was tested at the PAS ATTAAA sequence and this nuclease produced efficient editing in a B-LCL cell line and in a 15Abic FSHD patient myoblast line. See, FIG. 1A. In one embodiment, the present invention contemplates a therapeutic strategy comprising a Cas12a nuclease and adenine base editing (ABE) of the DUX4 polyadenylation sequence (PAS) to reduce disease-causing DUX4 mRNA. See FIG. 1B.

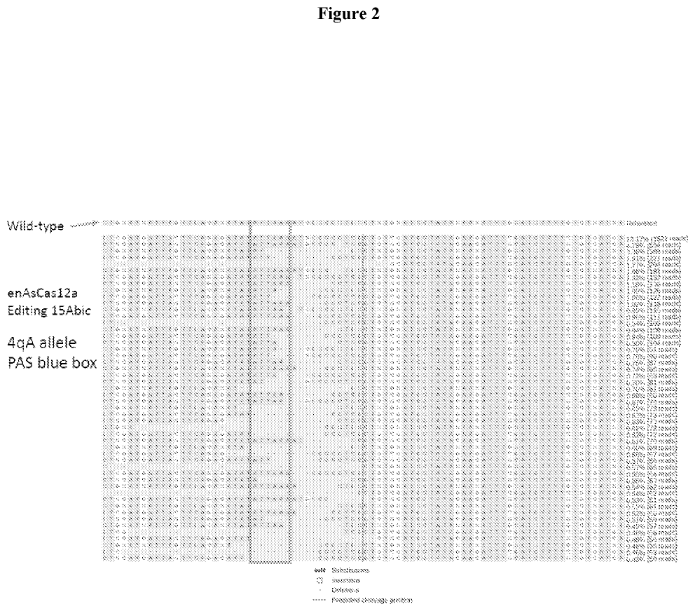

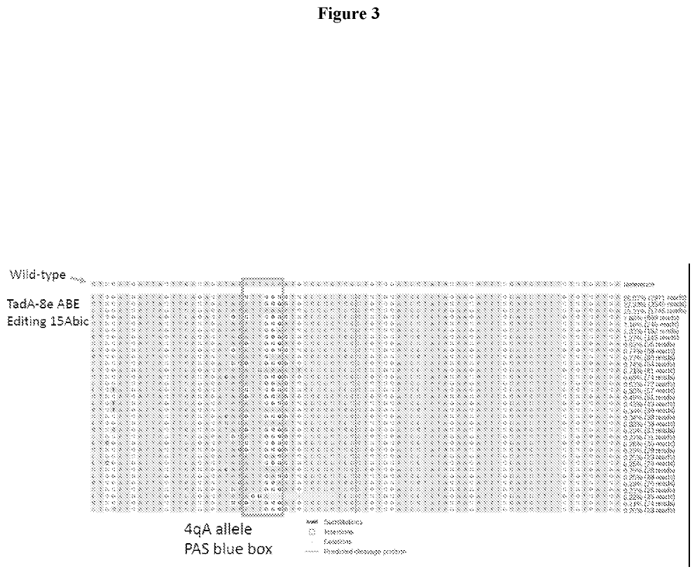

Both the 3xNLS-enAsCas12a nuclease and the SpCas9-TadA8e adenine base editor produced highly efficient editing in the 15Abic FSHD patient myoblast line that results in the disruption of the 4qA PAS sequence (>80%) based on targeted deep sequencing of PCR amplicons spanning the target site on Chromosome 4. See, FIGS. 2 and 3.

### B. Gene Editing of Muscular Dystrophy Cells

In one embodiment, the present invention contemplates a method providing a muscle cell or satellite cell comprising a genetic mutation and/or pathogenic variant, wherein the muscle cell or satellite cell exhibits at least one symptom of muscular dystrophy. In one embodiment, the genetic mutation and/or pathogenic variant is the 4qA locus. In one embodiment, the pathogenic variant is an ATTAAA sequence. In one embodiment, the method further provides an enAsCas12a nuclease comprising a crRNA3 molecule or a TadA-8e ABE comprising a sgRNA 1 molecule. In one embodiment, the method further comprises administering the enAsCas12a nuclease or a TadA-8e ABE into the muscle cell or satellite cell. In one embodiment, the method further comprises editing the pathogenic variant with the enAsCas12a nuclease or a TadA-8e ABE, wherein the muscle cell does not exhibit the at least one symptom of muscular dystrophy. In one embodiment, the edited muscle cell or satellite cell grows into a differentiated muscle tissue. In one embodiment, the muscle cells are primary myoblasts isolated from the patient, edited ex vivo and then engrafted into the patient. In one embodiment, the muscle cell is an immortalized FSHD patient myoblast cell line. In one embodiment, the immortalized FSHD patient myoblast cell line is a 15Abic cell line. In one embodiment, the muscle cell is derived from an FSHD mouse. In one embodiment, the FSHD mouse is an FLExDUX4 mouse. Jones et al., PLoS One 13(2):e0192657 (2018). In one embodiment, the muscle cell, myoblast cell or satellite cell is derived from a human patient diagnosed with muscular dystrophy. In one embodiment, the muscular dystrophy is facioscapulohumeral muscular dystrophy.

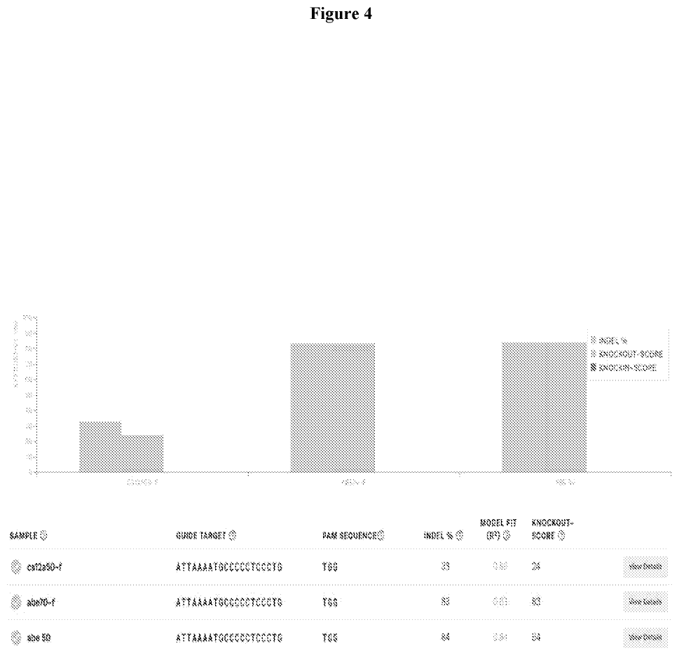

Primary IL 17A-myoblasts were subjected to gene editing using an adenosine base editors at two different amounts (50 pmol and 70 pmol) that were compared with a Cas12a nuclease in accordance with Example I. The data shows that when sharing the same guide target and PAM, the gene editing activity of the two adenosine base editors were approximately 4-fold greater than the Cas12 nuclease. In particular, Cas12a and ABE editing in FSHD patient primary myoblasts cause 30% and 70% disruption of the DUX4 PAS sequence, respectively. See, FIG. 4.

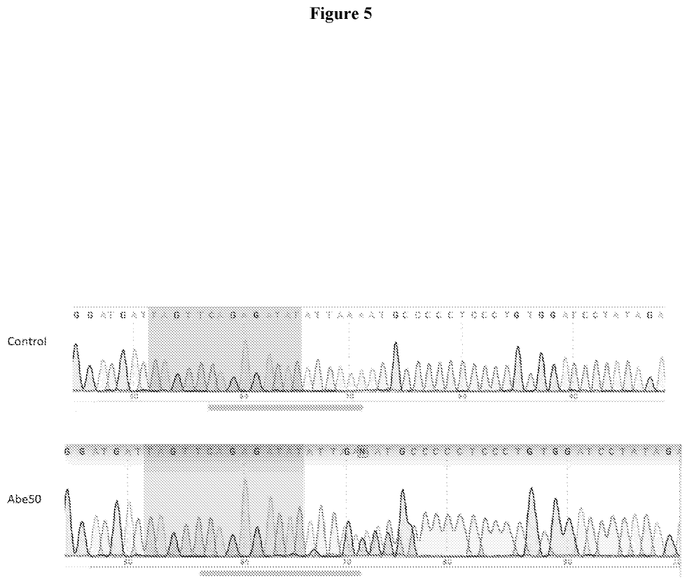

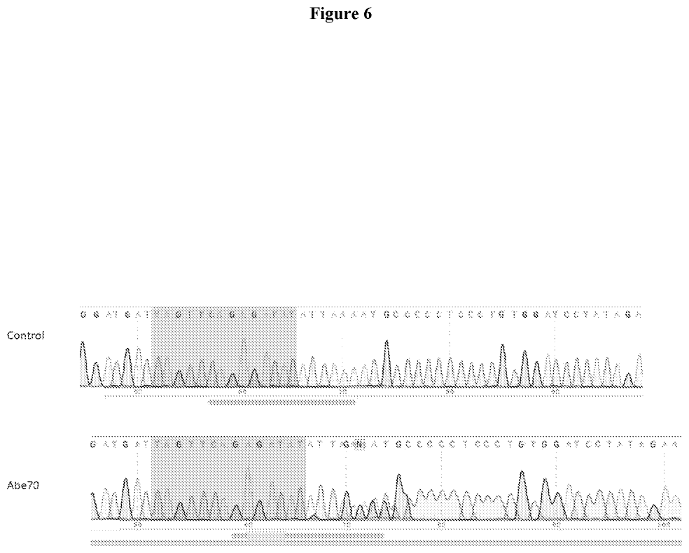

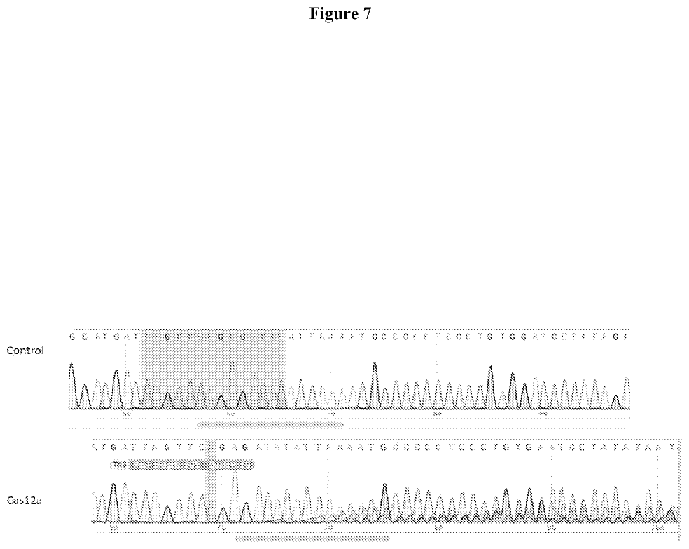

A Sanger sequencing analysis identified the specific edited bases for each nuclease. FSHD patient primary myoblasts treated with 50 pmol ABE protein show robust A to G base conversion at the DUX4 4qA PAS sequence. See, FIG. 5. FSHD patient primary myoblasts treated with 70 pmol ABE protein show robust A to G conversion at the DUX4 4qA PAS sequence. See, FIG. 6. FSHD patient primary myoblasts treated with 80 pmol enCas12a nuclease show efficient indel editing at the DUX4 4qA PAS sequence. See, FIG. 7.

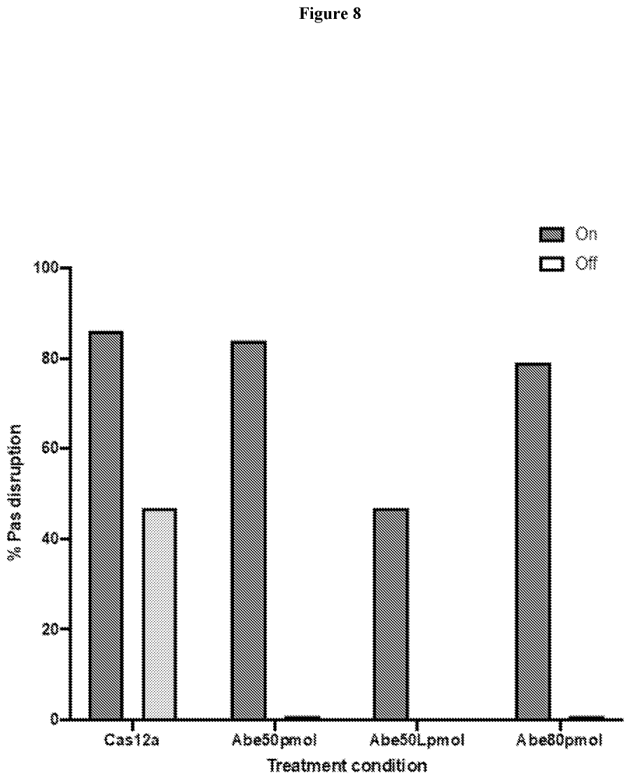

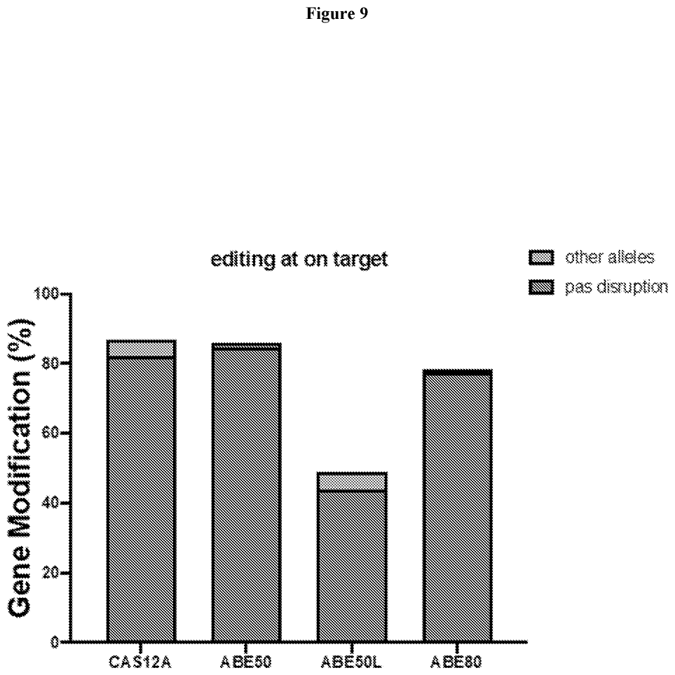

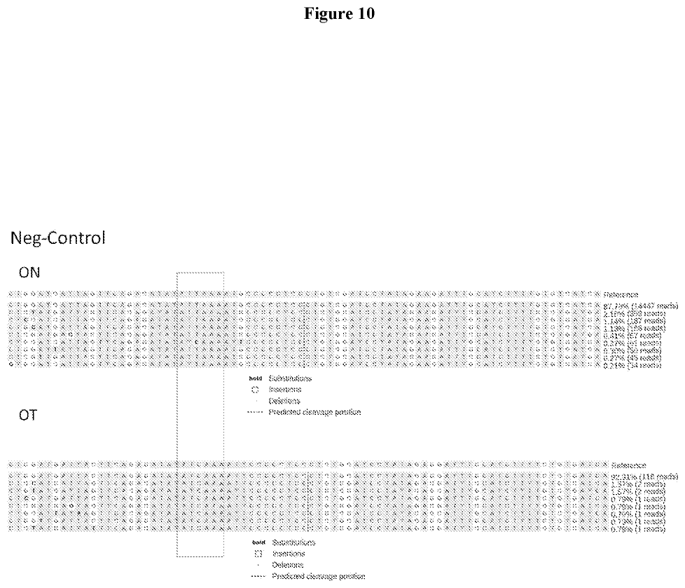

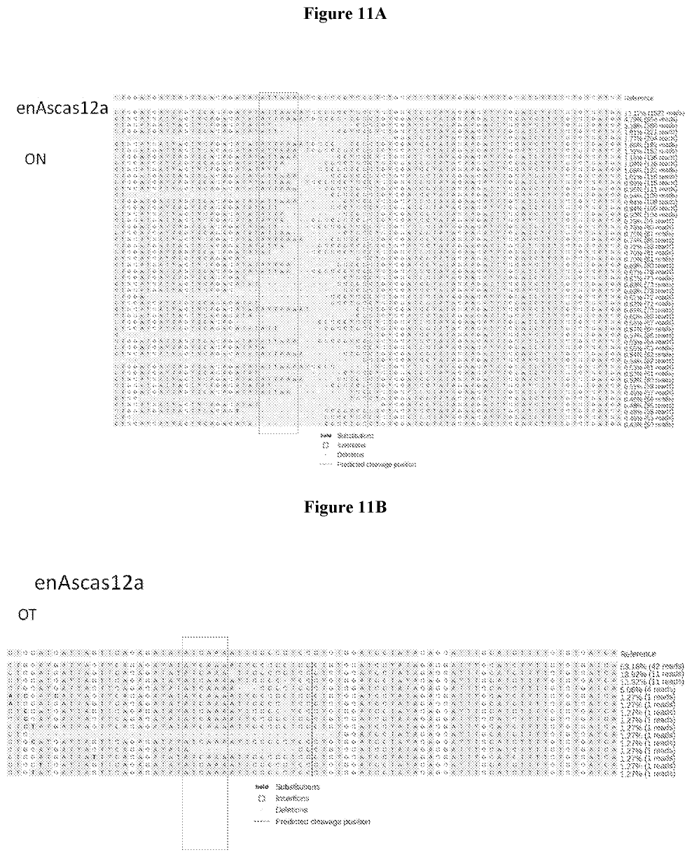

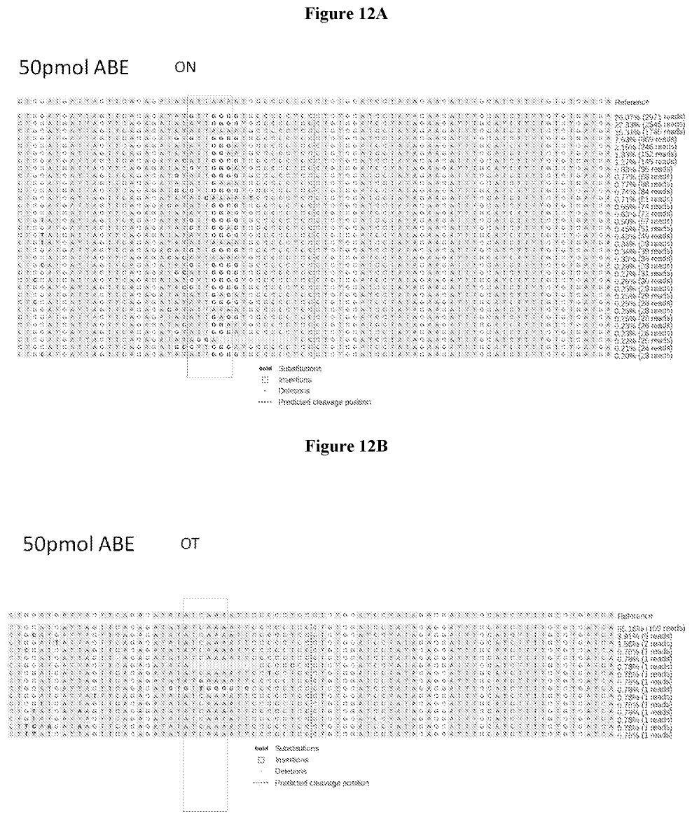

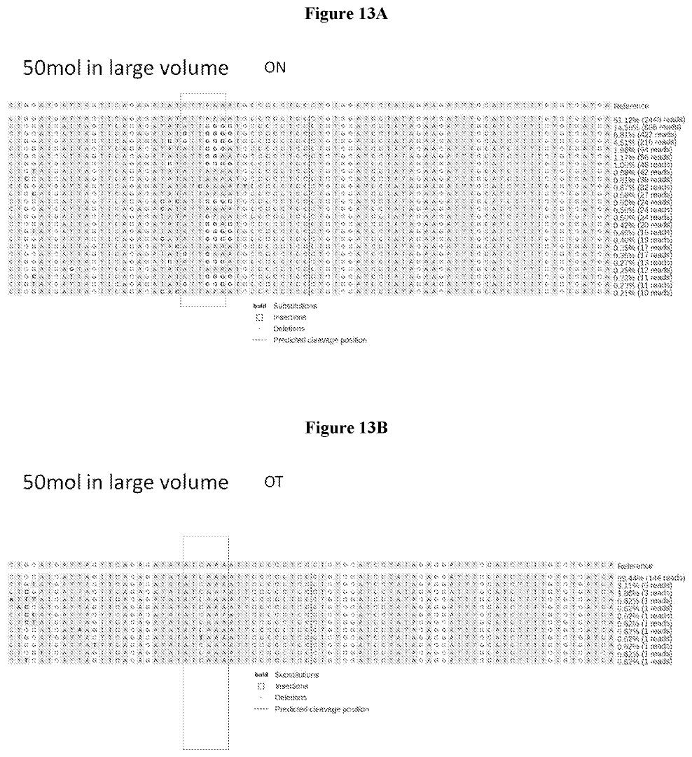

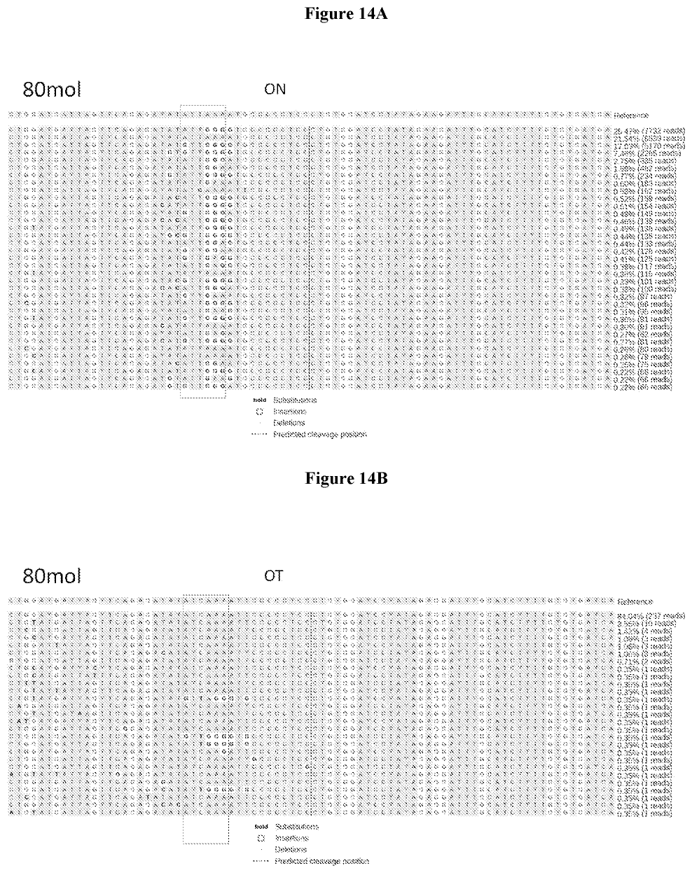

The gene editing activity of adenosine base editors (e.g., 50 pmol; Abe50, 50 pmol in a large volume; Abe50L and 80 pmol; Abe80) and a Cas12a nuclease was further investigated to determine their relative on/off target efficiencies in accordance with Example II. The data show on target editing rates are high for Cas12a and ABE DUX4 PAS editing, and 10qA off-target rates much lower for ABE editing. See, FIG. 8. The on-target editing (i.e., within the polyadenylation signal sequence; PAS) was observed to account for the vast majority of gene editing activity in all the tested nucleases. This majority of Cas12a and ABE edited alleles have sequence modifications in the 4qA DUX4 PAS sequence. See, FIG. 9. A deep sequencing analysis shows the specific edited bases that account for these observations; i) a negative control using untreated control myoblasts showing 4qA and 10qA DUX4 reference sequences (See, FIG. 10); ii) on-target analysis of enAscas12a-treated myoblasts show efficient editing at the 4qA DUX4 PAS sequence (See, FIG. 11A); iii) off-target analysis of enAscas12a treated cells have InDels at the 10qA locus, which has significant sequence similarity to 4qA DUX4 PAS sequence (See, FIG. 11B); iv) on-target analysis of myoblasts treated with 50 pmol ABE show efficient conversion of A's to G's at the DUX4 PAS sequence (See, FIG. 12A); v) off-target analysis of myoblasts treated with 50 pmol ABE have a small number of InDels at the 10qA locus, which has significant sequence similarity to 4qA DUX4 PAS sequence (See, FIG. 12B); vi) on-target analysis of myoblasts treated with a large volume of 50 pmol ABE (ABE50L) show efficient conversion of A's to G's at the 4qA DUX4 PAS sequence (See, FIG. 13A); vii) off-target analysis of myoblasts treated with a large volume of 50 pmol ABE have only modest number of InDels at the 10qA locus which has significant sequence similarity to 4qA DUX4 PAS sequence (See, FIG. 13B); and viii) on-target analysis of myoblasts treated with 80 pmol ABE (ABE80) show efficient conversion of A's to G's at the 4qA DUX4 PAS sequence (See, FIG. 14A); and ix) off-target analysis of myoblasts treated with 80 pmol ABE have a small number of InDels at the 10qA locus which has significant sequence similarity to 4qA DUX4 PAS sequence (See, FIG. 14B).

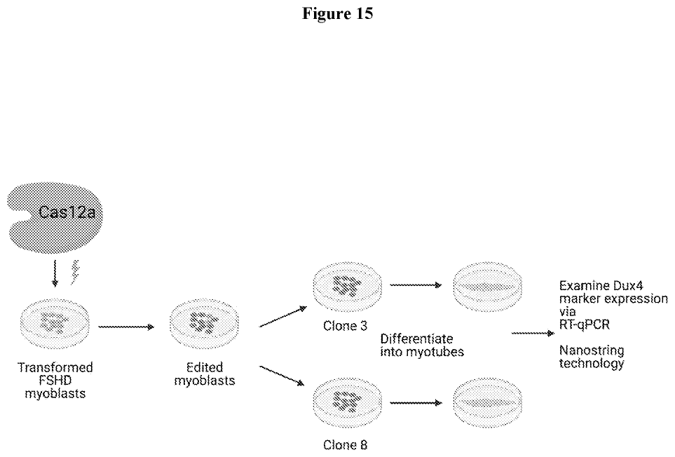

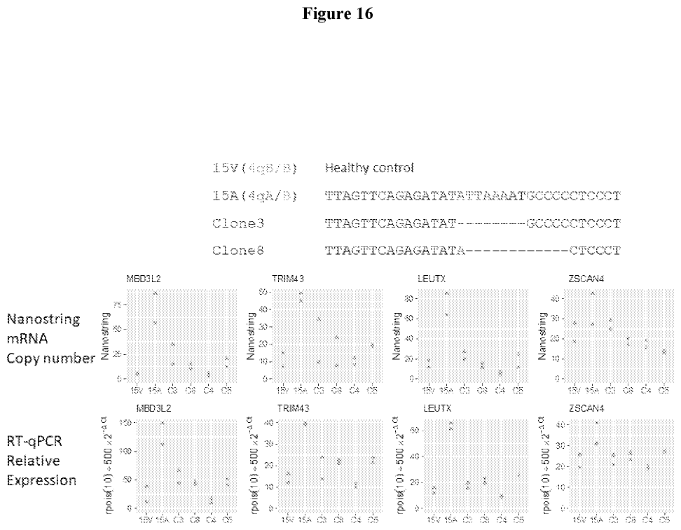

These edited PAS gene sequences were evaluated using a clonal analysis for a downstream effect on DUX4 markers (e.g., MBD3L2, TRIM43, LEUTX and ZSCAN4). The protocol addresses the question as to whether DUX4 PAS editing reduces DUX4 mRNA in FSHD patient myoblasts. The schematic shows a method for generating myoblast clonal lines with unique edits within the PAS sequence of the 4qA DUX4 locus and then differentiation into myotubes for assessment of expression of Dux4 biomarker RNAs. 15Abic cells were treated with enAsCas12a RNPs targeting the PAS sequence within the DUX4 4qA locus. Clone 3 and clone 8 are clonal myoblast lines with disruptions in the PAS sequence. See, FIG. 15. The data show that DUX4 PAS editing reduces expression of DUX4 biomarkers. See, FIG. 16. These data strongly suggest that PAS gene editing modulates DUX4 gene expression would be expected to have therapeutic efficacy.

### III. Polymeric Delivery Systems

The present invention contemplates several drug delivery systems that provide for roughly uniform distribution, have controllable rates of release. A variety of different media are described below that are useful in creating drug delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

One embodiment of the present invention contemplates a drug delivery system comprising therapeutic agents as described herein.

### Microparticles

One embodiment of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

### Liposomes

One embodiment of the present invention contemplates liposomes capable of attaching and releasing therapeutic agents described herein. Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In some embodiments, the present invention contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes that provide controlled release of at least one therapeutic agent. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### Microspheres, Microparticles and Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze dried medium comprising at least one therapeutic agent is homogenized in a suitable solvent and sprayed to manufacture microspheres in the range of 20 to 90 μm. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of therapeutic agent release. Miller et al., Degradation Rates of Oral Resorbable Implants {Polylactates and Polyglycolates: Rate Modification and Changes in PLA/PGA Copolymer Ratios, J. Biomed. Mater. Res., Vol. II:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a therapeutic agent is added to the biodegradable polymer metal salt solution. The weight ratio of a therapeutic agent to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and therapeutic agent is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a therapeutic agent for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-500 μm and composed of biocompatible and biodegradable polymers. Specific polymer compositions of a microsphere can control the therapeutic agent release rate such that custom-designed microspheres are possible, including effective management of the burst effect. ProMaxx® (Epic Therapeutics, Inc.) is a protein-matrix delivery system. The system is aqueous in nature and is adaptable to standard pharmaceutical delivery models. In particular, ProMaxx® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a therapeutic agent is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one embodiment, the present invention contemplates microparticles formed by spray-drying a composition comprising fibrinogen or thrombin with a therapeutic agent. Preferably, these microparticles are soluble and the selected protein (i.e., fibrinogen or thrombin) creates the walls of the microparticles. Consequently, the therapeutic agents are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

### IV. Associated Adenoviral Delivery Systems

Adeno-associated virus (AAV) is a replication-deficient parvovirus, the single-stranded DNA genome of which is about 4.7 kb in length including 145 nucleotide inverted terminal repeat (ITRs). The nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56.degree. to 65.degree. C. for several hours), making cold preservation of AAV less critical. AAV may even be lyophilized. Finally, AAV-infected cells are not resistant to superinfection. Mendell et al., “Guided injections for AAV gene transfer to muscle” U.S. Pat. No. 10,842,886 (herein incorporated by reference).

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

### EXPERIMENTAL

### Example 1

### Comparative Gene Editing Activity

This example presents a protocol for assessing gene editing in primary myoblasts, whether the myoblasts are derived from a healthy donor or a donor exhibiting muscular dystrophy.

Primary IL 17A-myoblasts, having a 4qA/4qA genotype, were cultured to a 50-70% confluence of 50 and 70% over a period of ˜2 weeks. An aliquot of approximately 100,000 cultured myoblasts were then electroporated with either 100 pmol an adenosine base editor (AbE) or 80 pmols of an enCas12a nuclease.

The myoblasts were then cultured to confluence and harvested. Genomic DNA was extracted and analyzed for editing at the polyadenylation signal (PAS) sequence by known deep sequencing techniques.

### Example II

### On/Off-Target Gene Editing

This example presents a protocol for assessing on/off-target gene editing in cells, whether the cells are derived from a healthy donor or a donor exhibiting muscular dystrophy.

Briefly, IL-15Abic cells were cultured to 60-70% confluence and treated with: i) 80 pmol Cas12a/240 pmol of guide (Cas12a); ii) 50 pmol ABE/150 pmol guide (ABE50); iii) 50 pmol ABE/150 pmol guide in 12 ul neon buffer (ABE50L); and iv) 80 pmol ABE/240 pmol guide (ABE80).

The 15Abic cells were harvested after three 3 days of nuclease incubation. Genomic DNA was extracted and on/off target gene editing was determined using a known deep sequencing analysis. Due to the extent of sequence similarity with a Chromosome 10 region observed single nucleotide polymorphisms were interpreted as “off-target” gene editing.

### Example III

### Nucleic Acid Analysis of DUX4 Expression Markers

This example describes a protocol to ascertain modulation of DUX4 expression by measuring downstream gene expression of MBD3L2, TRIM43, LEUTX and ZSCAN4 which are known to modulated by DUX4.

Briefly, a cell culture techniques was performed as illustrated in FIG. 15. After myotube differentiation, the DNA was extracted and MBD3L2, TRIM43, LEUTX and ZSCAN4 mRNA levels were measured by known nanostring and RT-qPCR techniques.

1. A method, comprising:
a) providing;
i) a mammal exhibiting at least one symptom of a muscular dystrophy disease and a genetic mutation and/or pathogenic variant;
ii) a nuclease or base-modifying protein targeted to said genetic mutation or pathogenic variant; and
b) editing said genetic mutation such that said at least one symptom is reduced.

2. The method of claim 1, wherein said genetic mutation and/or pathogenic variant is in a muscle cell.

3. The method of claim 1, wherein said pathogenic variant is the 4qA locus and comprises an ATTAAA sequence.

4. (canceled)

5. The method of claim 1, wherein said genetic mutation is within a nucleic acid sequence of a gene selected from the group consisting of a DMD gene, a COL6A gene, a DYSF gene, an ANO5 gene, an EMD gene, an LMNA gene, a DUX4 gene, a DYSF gene, a DMPK gene, a ZNF9 (CNBP) gene and/or a PABPN1 gene.

6. The method of claim 1, wherein said nuclease or base-modifying protein is selected from the group consisting of an enAsCas12a nuclease, an adenine base editor and a primer editor.

7. The method of claim 6, wherein said enAsCas12a nuclease binds to a crRNA3 molecule.

8-9. (canceled)

10. The method of claim 6, wherein said adenine base editor is a TadA-8e adenine base editor.

11. The method of claim 2, wherein said method further comprises administering said nuclease or base-modifying protein into said muscle cell to create an edited muscle cell.

12. The method of claim 11, wherein said method further comprises differentiating said edited muscle cell into a muscle tissue.

13. The method of claim 2, wherein said muscle cell is selected from the group consisting of an in vivo human muscle cell, an in vitro human muscle cells, a primary patient myoblast and an FSHD mouse muscle cell.

14. (canceled)

15. The method of claim 13, wherein said in vitro human muscle cell line is an immortalized FSHD patient myoblast cell line.

16-18. (canceled)

19. The method of claim 1, wherein said muscular dystrophy is facioscapulohumeral muscular dystrophy.

20. The method of claim 11, wherein said administering further comprises a nanoparticle comprising said nuclease or said base-modifying protein.

21. The method of claim 11, wherein said administering further comprises an associated adenovirus comprising a packaged genome encoding said nuclease or said base-modifying protein.

22. A nuclease or base editor protein targeted to a muscular dystrophy genetic mutation or pathogenic variation.

23. The nuclease protein of claim 22, wherein said nuclease protein is an enAsCas12a nuclease protein.

24. The nuclease protein of claim 23, wherein said enAsCas12a nuclease binds to a crRNA3 guide RNA.

25. The base editor protein of claim 22 wherein said base editor is an adenine base editor.

26. The nuclease protein of claim 25, wherein said adenine base editor is a TadA-8e adenine base editor that binds to an sgRNA1 guide RNA.

27. The nuclease or base editor protein of claim 22, wherein said muscular dystrophy genetic mutation or pathogenic variation is within a nucleic acid sequence of a gene selected from the group consisting of a DMD gene, a COL6A gene, a DYSF gene, an ANO5 gene, an EMD gene, an LMNA gene, a DUX4 gene, a DYSF gene, a DMPK gene, a ZNF9 (CNBP) gene and/or a PABPN1 gene.


SEQUENCE LISTING (323 sequences)

SEQ ID NO: 1 - protein, 7 aa, Artificial Sequence
  Synthetic
PKKKRKV

SEQ ID NO: 2 - protein, 18 aa, Artificial Sequence
  Synthetic
KRTADGSEFESPKKKRKV

SEQ ID NO: 3 - protein, 18 aa, Artificial Sequence
  Synthetic
KRTADSSHSTPPKTKRKV

SEQ ID NO: 4 - protein, 18 aa, Artificial Sequence
  Synthetic
KRPAATKKAGQAKKKKLD

SEQ ID NO: 5 - protein, 9 aa, Artificial Sequence
  Synthetic
PAAKRVKLD

SEQ ID NO: 6 - DNA, 53 bp, Artificial Sequence
  Synthetic
ggatgattagttcagagatatattaaaatgccccctccctgtggatcctatag

SEQ ID NO: 7 - DNA, 53 bp, Artificial Sequence; identical to SEQ ID NO: 6
  Synthetic

SEQ ID NO: 8 - DNA, 38 bp, Artificial Sequence
  Synthetic
gagatatattaaaatgccccctccctgtggatcctata

SEQ ID NO: 9 - DNA, 80 bp, Artificial Sequence
  Synthetic
ctggatgattagttcagagatatattaaaatgccccctccctgtggatcctatagaagatttgcatcttttgtgtgatga

SEQ ID NO: 10 - DNA, 80 bp, Artificial Sequence
  Synthetic
  misc_feature (37)..(37): n is a, c, g, or t
ctggatgattagttcagagatatattaaaatgccccntccctgtggatcctatagaagatttgcatcttttgtgtgatga

SEQ ID NO: 11 - DNA, 80 bp, Artificial Sequence
  Synthetic
  misc_feature (26)..(38): n is a, c, g, or t
ctggatgattagttcagagatatatnnnnnnnnnnnnnccctgtggatcctatagaagatttgcatcttttgtgtgatga

SEQ ID NO: 12 - DNA, 80 bp, Artificial Sequence
  Synthetic
  misc_feature (27)..(35): n is a, c, g, or t
ctggatgattagttcagagatatattnnnnnnnnncctccctgtggatcctatagaagatttgcatcttttgtgtgatga

SEQ ID NO: 13 - DNA, 80 bp, Artificial Sequence
  Synthetic
  misc_feature (1)..(44): n is a, c, g, or t
nnnnnnnnnnnnnnnnnnnnnnnnnnnnnnnnnnnnnnnnnnnnggatcctatagaagatttgcatcttttgtgtgatga

SEQ ID NO: 14 - DNA, 80 bp, Artificial Sequence
  Synthetic
  misc_feature (36)..(37): n is a, c, g, or t
ctggatgattagttcagagatatattaaaatgcccnntccctgtggatcctatagaagatttgcatcttttgtgtgatga

SEQ ID NO: 15 - DNA, 80 bp, Artificial Sequence
  Synthetic
  misc_feature (30)..(30): n is a, c, g, or t
ctggatgattagttcagagatatattaaantgccccctccctgtggatcctatagaagatttgcatcttttgtgtgatga

SEQ ID NO: 16 - DNA, 80 bp, Artificial Sequence
  Synthetic
  misc_feature (30)..(35): n is a, c, g, or t
ctggatgattagttcagagatatattaaannnnnncctccctgtggatcctatagaagatttgcatcttttgtgtgatga

SEQ ID NO: 17 - DNA, 80 bp, Artificial Sequence
  Synthetic
  misc_feature (28)..(33): n is a, c, g, or t
ctggatgattagttcagagatatattannnnnncccctccctgtggatcctatagaagatttgcatcttttgtgtgatga

SEQ ID NO: 18 - DNA, 80 bp, Artificial Sequence
  Synthetic
  misc_feature (27)..(32): n is a, c, g, or t
ctggatgattagttcagagatatattnnnnnnccccctccctgtggatcctatagaagatttgcatcttttgtgtgatga

SEQ ID NO: 19 - DNA, 80 bp, Artificial Sequence
  Synthetic
  misc_feature (30)..(34): n is a, c, g, or t
ctggatgattagttcagagatatattaaannnnnccctccctgtggatcctatagaagatttgcatcttttgtgtgatga

SEQ ID NO: 20 - DNA, 80 bp, Artificial Sequence
  Synthetic
  misc_feature (29)..(30): n is a, c, g, or t
ctggatgattagttcagagatatattaanntgccccctccctgtggatcctatagaagatttgcatcttttgtgtgatga

SEQ ID NO: 21 - DNA, 80 bp, Artificial Sequence
  Synthetic
  misc_feature (30)..(33): n is a, c, g, or t
ctggatgattagttcagagatatattaaannnncccctccctgtggatcctatagaagatttgcatcttttgtgtgatga

SEQ ID NO: 22 - DNA, 80 bp, Artificial Sequence
  Synthetic
  misc_feature (3)..(34): n is a, c, g, or t
ctnnnnnnnnnnnnnnnnnnnnnnnnnnnnnnnnccctccctgtggatcctatagaagatttgcatcttttgtgtgatga

SEQ ID NO: 23 - DNA, 80 bp, Artificial Sequence
  Synthetic
  misc_feature (30)..(36): n is a, c, g, or t
ctggatgattagttcagagatatattaaannnnnnnctccctgtggatcctatagaagatttgcatcttttgtgtgatga

SEQ ID NO: 24 - DNA, 80 bp, Artificial Sequence
  Synthetic
  misc_feature (27)..(34): n is a, c, g, or t
ctggatgattagttcagagatatattnnnnnnnnccctccctgtggatcctatagaagatttgcatcttttgtgtgatga

SEQ ID NO: 25 - DNA, 80 bp, Artificial Sequence
  Synthetic
  misc_feature (28)..(36): n is a, c, g, or t
ctggatgattagttcagagatatattannnnnnnnnctccctgtggatcctatagaagatttgcatcttttgtgtgatga

SEQ ID NO: 26 - DNA, 80 bp, Artificial Sequence
  Synthetic
  misc_feature (28)..(31): n is a, c, g, or t
ctggatgattagttcagagatatattannnngccccctccctgtggatcctatagaagatttgcatcttttgtgtgatga

SEQ ID NO: 27 - DNA, 80 bp, Artificial Sequence
  Synthetic
  misc_feature (27)..(31): n is a, c, g, or t
ctggatgattagttcagagatatattnnnnngccccctccctgtggatcctatagaagatttgcatcttttgtgtgatga

SEQ ID NO: 28 - DNA, 80 bp, Artificial Sequence
  Synthetic
  misc_feature (29)..(36): n is a, c, g, or t
ctggatgattagttcagagatatattaannnnnnnnctccctgtggatcctatagaagatttgcatcttttgtgtgatga

SEQ ID NO: 29 - DNA, 80 bp, Artificial Sequence
  Synthetic
  misc_feature (31)..(34): n is a, c, g, or t
ctggatgattagttcagagatatattaaaannnnccctccctgtggatcctatagaagatttgcatcttttgtgtgatga

SEQ ID NO: 30 - DNA, 80 bp, Artificial Sequence
  Synthetic
  misc_feature (28)..(32): n is a, c, g, or t
ctggatgattagttcagagatatattannnnnccccctccctgtggatcctatagaagatttgcatcttttgtgtgatga

SEQ ID NO: 31 - DNA, 80 bp, Artificial Sequence
  Synthetic
  misc_feature (26)..(31): n is a, c, g, or t
ctggatgattagttcagagatatatnnnnnngccccctccctgtggatcctatagaagatttgcatcttttgtgtgatga

SEQ ID NO: 32 - DNA, 80 bp, Artificial Sequence
  Synthetic
  misc_feature (22)..(38): n is a, c, g, or t
ctggatgattagttcagagatnnnnnnnnnnnnnnnnnccctgtggatcctatagaagatttgcatcttttgtgtgatga

SEQ ID NO: 33 - DNA, 80 bp, Artificial Sequence
  Synthetic
  misc_feature (29)..(35): n is a, c, g, or t
ctggatgattagttcagagatatattaannnnnnncctccctgtggatcctatagaagatttgcatcttttgtgtgatga

SEQ ID NO: 34 - DNA, 80 bp, Artificial Sequence
  Synthetic
  misc_feature (29)..(31): n is a, c, g, or t
ctggatgattagttcagagatatattaannngccccctccctgtggatcctatagaagatttgcatcttttgtgtgatga

SEQ ID NO: 35 - DNA, 80 bp, Artificial Sequence
  Synthetic
  misc_feature (22)..(36): n is a, c, g, or t
ctggatgattagttcagagatnnnnnnnnnnnnnnnctccctgtggatcctatagaagatttgcatcttttgtgtgatga

SEQ ID NO: 36 - DNA, 80 bp, Artificial Sequence
  Synthetic
  misc_feature (24)..(35): n is a, c, g, or t
ctggatgattagttcagagatatnnnnnnnnnnnncctccctgtggatcctatagaagatttgcatcttttgtgtgatga

SEQ ID NO: 37 - DNA, 80 bp, Artificial Sequence
  Synthetic
  misc_feature (20)..(36): n is a, c, g, or t
ctggatgattagttcagagnnnnnnnnnnnnnnnnnctccctgtggatcctatagaagatttgcatcttttgtgtgatga

SEQ ID NO: 38 - DNA, 80 bp, Artificial Sequence
  Synthetic
  misc_feature (6)..(34): n is a, c, g, or t
ctggannnnnnnnnnnnnnnnnnnnnnnnnnnnnccctccctgtggatcctatagaagatttgcatcttttgtgtgatga

SEQ ID NO: 39 - DNA, 80 bp, Artificial Sequence
  Synthetic
  misc_feature (24)..(34): n is a, c, g, or t
ctggatgattagttcagagatatnnnnnnnnnnnccctccctgtggatcctatagaagatttgcatcttttgtgtgatga

SEQ ID NO: 40 - DNA, 80 bp, Artificial Sequence
  Synthetic
  misc_feature (32)..(38): n is a, c, g, or t
ctggatgattagttcagagatatattaaaatnnnnnnnccctgtggatcctatagaagatttgcatcttttgtgtgatga

SEQ ID NO: 41 - DNA, 80 bp, Artificial Sequence
  Synthetic
  misc_feature (23)..(38): n is a, c, g, or t
ctggatgattagttcagagatannnnnnnnnnnnnnnnccctgtggatcctatagaagatttgcatcttttgtgtgatga

SEQ ID NO: 42 - DNA, 80 bp, Artificial Sequence
  Synthetic
  misc_feature (20)..(32): n is a, c, g, or t
ctggatgattagttcagagnnnnnnnnnnnnnccccctccctgtggatcctatagaagatttgcatcttttgtgtgatga

SEQ ID NO: 43 - DNA, 80 bp, Artificial Sequence
  Synthetic
  misc_feature (27)..(33): n is a, c, g, or t
ctggatgattagttcagagatatattnnnnnnncccctccctgtggatcctatagaagatttgcatcttttgtgtgatga

SEQ ID NO: 44 - DNA, 80 bp, Artificial Sequence
  Synthetic
  misc_feature (2)..(36): n is a, c, g, or t
cnnnnnnnnnnnnnnnnnnnnnnnnnnnnnnnnnnnctccctgtggatcctatagaagatttgcatcttttgtgtgatga

SEQ ID NO: 45 - DNA, 80 bp, Artificial Sequence
  Synthetic
  misc_feature (30)..(32): n is a, c, g, or t
ctggatgattagttcagagatatattaaannnccccctccctgtggatcctatagaagatttgcatcttttgtgtgatga

SEQ ID NO: 46 - DNA, 80 bp, Artificial Sequence
  Synthetic
  misc_feature (25)..(38): n is a, c, g, or t
ctggatgattagttcagagatatannnnnnnnnnnnnnccctgtggatcctatagaagatttgcatcttttgtgtgatga

SEQ ID NO: 47 - DNA, 80 bp, Artificial Sequence
  Synthetic
  misc_feature (31)..(36): n is a, c, g, or t
ctggatgattagttcagagatatattaaaannnnnnctccctgtggatcctatagaagatttgcatcttttgtgtgatga

SEQ ID NO: 48 - DNA, 80 bp, Artificial Sequence
  Synthetic
  misc_feature (1)..(39): n is a, c, g, or t
nnnnnnnnnnnnnnnnnnnnnnnnnnnnnnnnnnnnnnncctgtggatcctatagaagatttgcatcttttgtgtgatga

SEQ ID NO: 49 - DNA, 80 bp, Artificial Sequence
  Synthetic
  misc_feature (36)..(41): n is a, c, g, or t
ctggatgattagttcagagatatattaaaatgcccnnnnnntgtggatcctatagaagatttgcatcttttgtgtgatga

SEQ ID NO: 50 - DNA, 80 bp, Artificial Sequence
  Synthetic
  misc_feature (24)..(36): n is a, c, g, or t
ctggatgattagttcagagatatnnnnnnnnnnnnnctccctgtggatcctatagaagatttgcatcttttgtgtgatga

SEQ ID NO: 51 - DNA, 80 bp, Artificial Sequence
  Synthetic
  misc_feature (31)..(35): n is a, c, g, or t
ctggatgattagttcagagatatattaaaannnnncctccctgtggatcctatagaagatttgcatcttttgtgtgatga

SEQ ID NO: 52 - DNA, 80 bp, Artificial Sequence
  Synthetic
  misc_feature (26)..(34): n is a, c, g, or t
ctggatgattagttcagagatatatnnnnnnnnnccctccctgtggatcctatagaagatttgcatcttttgtgtgatga

SEQ ID NO: 53 - DNA, 80 bp, Artificial Sequence
  Synthetic
  misc_feature (29)..(37): n is a, c, g, or t
ctggatgattagttcagagatatattaannnnnnnnntccctgtggatcctatagaagatttgcatcttttgtgtgatga

SEQ ID NO: 54 - DNA, 80 bp, Artificial Sequence
  Synthetic
  misc_feature (5)..(37): n is a, c, g, or t
ctggnnnnnnnnnnnnnnnnnnnnnnnnnnnnnnnnntccctgtggatcctatagaagatttgcatcttttgtgtgatga

SEQ ID NO: 55 - DNA, 80 bp, Artificial Sequence
  Synthetic
  misc_feature (24)..(42): n is a, c, g, or t
ctggatgattagttcagagatatnnnnnnnnnnnnnnnnnnngtggatcctatagaagatttgcatcttttgtgtgatga

SEQ ID NO: 56 - DNA, 80 bp, Artificial Sequence
  Synthetic
  misc_feature (21)..(33): n is a, c, g, or t
ctggatgattagttcagagtnnnnnnnnnnnnncccctccctgtggatcctatagaagatttgcatcttttgtgtgatga

SEQ ID NO: 57 - DNA, 80 bp, Artificial Sequence
  Synthetic
  misc_feature (4)..(33): n is a, c, g, or t
ctgnnnnnnnnnnnnnnnnnnnnnnnnnnnnnncccctccctgtggatcctatagaagatttgcatcttttgtgtgatga

SEQ ID NO: 58 - DNA, 80 bp, Artificial Sequence
  Synthetic
  misc_feature (23)..(33): n is a, c, g, or t
ctggatgattagttcagagatannnnnnnnnnncccctccctgtggatcctatagaagatttgcatcttttgtgtgatga

SEQ ID NO: 59 - DNA, 80 bp, Artificial Sequence
  Synthetic
ctggatgattagttcagagatatgttggggtgccccctccctgtggatcctatagaagatttgcatcttttgtgtgatga

SEQ ID NO: 60 - DNA, 80 bp, Artificial Sequence
  Synthetic
ctggatgattagttcagagatatattggggtgccccctccctgtggatcctatagaagatttgcatcttttgtgtgatga

SEQ ID NO: 61 - DNA, 80 bp, Artificial Sequence; identical to SEQ ID NO: 9
  Synthetic

SEQ ID NO: 62 - DNA, 80 bp, Artificial Sequence
  Synthetic
ctggatgattagttcagagatgtgttggggtgccccctccctgtggatcctatagaagatttgcatcttttgtgtgatga

SEQ ID NO: 63 - DNA, 80 bp, Artificial Sequence
  Synthetic
ctggatgattagttcagagatatattgggatgccccctccctgtggatcctatagaagatttgcatcttttgtgtgatga

SEQ ID NO: 64 - DNA, 80 bp, Artificial Sequence
  Synthetic
ctggatgattagttcagagatatattggaatgccccctccctgtggatcctatagaagatttgcatcttttgtgtgatga

SEQ ID NO: 65 - DNA, 80 bp, Artificial Sequence
  Synthetic
ctggatgattagttcagagatacattggggtgccccctccctgtggatcctatagaagatttgcatcttttgtgtgatga

SEQ ID NO: 66 - DNA, 80 bp, Artificial Sequence
  Synthetic
ctggatgattagttcagagatatgttgggatgccccctccctgtggatcctatagaagatttgcatcttttgtgtgatga

SEQ ID NO: 67 - DNA, 80 bp, Artificial Sequence
  Synthetic
ctggatgattagttcagagatgtattggggtgccccctccctgtggatcctatagaagatttgcatcttttgtgtgatga

SEQ ID NO: 68 - DNA, 80 bp, Artificial Sequence
  Synthetic
ctggatgattagttcagagatatattgaaatgccccctccctgtggatcctatagaagatttgcatcttttgtgtgatga

SEQ ID NO: 69 - DNA, 80 bp, Artificial Sequence
  Synthetic
ctggatgattagttcagagatgcgttggggtgccccctccctgtggatcctatagaagatttgcatcttttgtgtgatga

SEQ ID NO: 70 - DNA, 80 bp, Artificial Sequence
  Synthetic
ctggatgattagttcagagatatatcaaaattccccctccctgtggatcctatagaagatttgcatcttttgtgtgatga

SEQ ID NO: 71 - DNA, 80 bp, Artificial Sequence
  Synthetic
ctggatgattagttcagagacatattggggtgccccctccctgtggatcctatagaagatttgcatcttttgtgtgatga

SEQ ID NO: 72 - DNA, 80 bp, Artificial Sequence
  Synthetic
ctggatgattagttcagagatatattggagtgccccctccctgtggatcctatagaagatttgcatcttttgtgtgatga

SEQ ID NO: 73 - DNA, 80 bp, Artificial Sequence
  Synthetic
ctgtatgattagttcagagatatgttggggtgccccctccctgtggatcctatagaagatttgcatcttttgtgtgatga

SEQ ID NO: 74 - DNA, 80 bp, Artificial Sequence
  Synthetic
ctggatgattagttcagagatatgttggagtgccccctccctgtggatcctatagaagatttgcatcttttgtgtgatga

SEQ ID NO: 75 - DNA, 80 bp, Artificial Sequence
  Synthetic
ctgtatgattagttcagagatatattggggtgccccctccctgtggatcctatagaagatttgcatcttttgtgtgatga

SEQ ID NO: 76 - DNA, 80 bp, Artificial Sequence
  Synthetic
ctgtatgattagttcagagatatattaaaatgccccctccctgtggatcctatagaagatttgcatcttttgtgtgatga

SEQ ID NO: 77 - DNA, 80 bp, Artificial Sequence
  Synthetic
ctggatgattagttcagagacatgttggggtgccccctccctgtggatcctatagaagatttgcatcttttgtgtgatga

SEQ ID NO: 78 - DNA, 80 bp, Artificial Sequence
  Synthetic
ctggatgattagttcagagacacattaaaatgccccctccctgtggatcctatagaagatttgcatcttttgtgtgatga

SEQ ID NO: 79 - DNA, 80 bp, Artificial Sequence
  Synthetic
ctgcatgattagttcagagatatgttggggtgccccctccctgtggatcctatagaagatttgcatcttttgtgtgatga

SEQ ID NO: 80 - DNA, 80 bp, Artificial Sequence
  Synthetic
ctggatgattagttcagagatgcattggggtgccccctccctgtggatcctatagaagatttgcatcttttgtgtgatga

SEQ ID NO: 81 - DNA, 80 bp, Artificial Sequence
  Synthetic
ctcgatgattagttcagagatatgttggggtgccccctccctgtggatcctatagaagatttgcatcttttgtgtgatga

SEQ ID NO: 82 - DNA, 80 bp, Artificial Sequence
  Synthetic
ctggatgattagttcagagatacgttggggtgccccctccctgtggatcctatagaagatttgcatcttttgtgtgatga

SEQ ID NO: 83 - DNA, 80 bp, Artificial Sequence
  Synthetic
ctggatgattagttcagagatatattgagatgccccctccctgtggatcctatagaagatttgcatcttttgtgtgatga

SEQ ID NO: 84 - DNA, 80 bp, Artificial Sequence
  Synthetic
ctcgatgattagttcagagatatattggggtgccccctccctgtggatcctatagaagatttgcatcttttgtgtgatga

SEQ ID NO: 85 - DNA, 80 bp, Artificial Sequence
  Synthetic
ctggatgattagttcagagacatattaaaatgccccctccctgtggatcctatagaagatttgcatcttttgtgtgatga

SEQ ID NO: 86 - DNA, 80 bp, Artificial Sequence
  Synthetic
ctggatgattagttcagagatatattgaggtgccccctccctgtggatcctatagaagatttgcatcttttgtgtgatga

SEQ ID NO: 87 - DNA, 80 bp, Artificial Sequence
  Synthetic
ctggatgattagttcagagatacattggaatgccccctccctgtggatcctatagaagatttgcatcttttgtgtgatga

SEQ ID NO: 88 - DNA, 80 bp, Artificial Sequence
  Synthetic
ctggatgattagttcagagatgtattgggatgccccctccctgtggatcctatagaagatttgcatcttttgtgtgatga

SEQ ID NO: 89 - DNA, 80 bp, Artificial Sequence
  Synthetic
  misc_feature (28)..(37): n is a, c, g, or t
ctggatgattagttcagagatataggannnnnnnnnntccctgtggatcctatagaagatttgcatcttttgtgtgatga

SEQ ID NO: 90 - DNA, 80 bp, Artificial Sequence
  Synthetic
ctggatgattagttcagagatgcgttgggatgccccctccctgtggatcctatagaagatttgcatcttttgtgtgatga

SEQ ID NO: 91 - DNA, 80 bp, Artificial Sequence
  Synthetic
ctgcatgattagttcagagatatattggggtgccccctccctgtggatcctatagaagatttgcatcttttgtgtgatga

SEQ ID NO: 92 - DNA, 20 bp, Artificial Sequence
  Synthetic
attaaaatgccccctccctg

SEQ ID NO: 93 - DNA, 54 bp, Artificial Sequence
  Synthetic
ggatgattagttcagagatatattaaaatgccccctccctgtggatcctataga

SEQ ID NO: 94 - DNA, 55 bp, Artificial Sequence
  Synthetic
  misc_feature (28)..(28): n is a, c, g, or t
ggatgattagttcagagatatattaganaatgccccctccctgtggatcctatag

SEQ ID NO: 95 - DNA, 56 bp, Artificial Sequence
  Synthetic
  misc_feature (27)..(27): n is a, c, g, or t
gatgattagttcagagatatattaganaatgccccctccctgtggatcctatagaa

SEQ ID NO: 96 - DNA, 54 bp, Artificial Sequence
  Synthetic
atgattagttcagagatatattaaaatgccccctccctgtgaatcctatataat

SEQ ID NO: 97 - skipped

SEQ ID NO: 98 - DNA, 80 bp, Artificial Sequence; identical to SEQ ID NO: 76
  Synthetic

SEQ ID NO: 99 - DNA, 80 bp, Artificial Sequence
  Synthetic
ctcgatgattagttcagagatatattaaaatgccccctccctgtggatcctatagaagatttgcatcttttgtgtgatga

SEQ ID NO: 100 - DNA, 80 bp, Artificial Sequence
  Synthetic
ctgcatgattagttcagagatatattaaaatgccccctccctgtggatcctatagaagatttgcatcttttgtgtgatga

SEQ ID NO: 101 - DNA, 80 bp, Artificial Sequence
  Synthetic
ctggatgagtagttcagagatatattaaaatgccccctccctgtggatcctatagaagatttgcatcttttgtgtgatga

SEQ ID NO: 102 - DNA, 80 bp, Artificial Sequence; identical to SEQ ID NO: 70
  Synthetic

SEQ ID NO: 103 - DNA, 80 bp, Artificial Sequence
  Synthetic
ctggattattagttcagagatatattaaaatgccccctccctgtggatcctatagaagatttgcatcttttgtgtgatga

SEQ ID NO: 104 - DNA, 80 bp, Artificial Sequence
  Synthetic
ctggttgattagttcagagatatattaaaatgccccctccctgtggatcctatagaagatttgcatcttttgtgtgatga

SEQ ID NO: 105 - DNA, 80 bp, Artificial Sequence
  Synthetic
gtggatgattagttcagagatatattaaaatgccccctccctgtggatcctatagaagatttgcatcttttgtgtgatga

SEQ ID NO: 106 - DNA, 80 bp, Artificial Sequence
  Synthetic
ctggatgattagttcagagatatatcaaaattccccctccctgtggatcctatagaggatttgcatcttttgtgtgatca

SEQ ID NO: 107 - DNA, 80 bp, Artificial Sequence; identical to SEQ ID NO: 106
  Synthetic

SEQ ID NO: 108 - DNA, 80 bp, Artificial Sequence
  Synthetic
ctgcatgattagttcagagatatatcaaaattccccctccctgtggatcctatagaggatttgcatcttttgtgtgatca

SEQ ID NO: 109 - DNA, 80 bp, Artificial Sequence
  Synthetic
ctgtatgattagttcagagatatatcaaaattccccctccctgtggatcctatagaggatttgcatcttttgtgtgatca

SEQ ID NO: 110 - DNA, 80 bp, Artificial Sequence
  Synthetic
ctcgatgattagttcagagatatatcaaaattccccctccctgtggatcctatagaggatttgcatcttttgtgtgatca

SEQ ID NO: 111 - DNA, 80 bp, Artificial Sequence
  Synthetic
ctggatgagtagttcagagatatatcaaaattccccctccctgtggatcctatagaggatttgcatcttttgtgtgatca

SEQ ID NO: 112 - DNA, 80 bp, Artificial Sequence
  Synthetic
ctggattataagttcagagatatatcaaaattccccctccctgtggatcctatagaggatttgcatcttttgtgtgatca

SEQ ID NO: 113 - DNA, 80 bp, Artificial Sequence
  Synthetic
ctggttgattagttcagagatatatcaaaattccccctccctgtggatcctatagaggatttgcatcttttgtgtgatca

SEQ ID NO: 114 - DNA, 80 bp, Artificial Sequence
  Synthetic
ctgtatgattatttcagagatatatcaaaattccccctccctgtggatcctatagaggatttgcatcttttgtgtgatca

SEQ ID NO: 115 - skipped

SEQ ID NO: 116 - DNA, 80 bp, Artificial Sequence; identical to SEQ ID NO: 10
  Synthetic
  misc_feature (37)..(37): n is a, c, g, or t

SEQ ID NO: 117 - DNA, 80 bp, Artificial Sequence; identical to SEQ ID NO: 11
  Synthetic
  misc_feature (26)..(38): n is a, c, g, or t

SEQ ID NO: 118 - DNA, 80 bp, Artificial Sequence; identical to SEQ ID NO: 12
  Synthetic
  misc_feature (27)..(35): n is a, c, g, or t

SEQ ID NO: 119 - DNA, 80 bp, Artificial Sequence; identical to SEQ ID NO: 13
  Synthetic
  misc_feature (1)..(44): n is a, c, g, or t

SEQ ID NO: 120 - DNA, 80 bp, Artificial Sequence; identical to SEQ ID NO: 14
  Synthetic
  misc_feature (36)..(37): n is a, c, g, or t

SEQ ID NO: 121 - DNA, 80 bp, Artificial Sequence; identical to SEQ ID NO: 15
  Synthetic
  misc_feature (30)..(30): n is a, c, g, or t

SEQ ID NO: 122 - DNA, 80 bp, Artificial Sequence; identical to SEQ ID NO: 16
  Synthetic
  misc_feature (30)..(35): n is a, c, g, or t

SEQ ID NO: 123 - DNA, 80 bp, Artificial Sequence; identical to SEQ ID NO: 17
  Synthetic
  misc_feature (28)..(33): n is a, c, g, or t

SEQ ID NO: 124 - DNA, 80 bp, Artificial Sequence; identical to SEQ ID NO: 18
  Synthetic
  misc_feature (27)..(32): n is a, c, g, or t

SEQ ID NO: 125 - DNA, 80 bp, Artificial Sequence; identical to SEQ ID NO: 19
  Synthetic
  misc_feature (30)..(34): n is a, c, g, or t

SEQ ID NO: 126 - DNA, 80 bp, Artificial Sequence; identical to SEQ ID NO: 20
  Synthetic
  misc_feature (29)..(30): n is a, c, g, or t

SEQ ID NO: 127 - DNA, 80 bp, Artificial Sequence; identical to SEQ ID NO: 21
  Synthetic
  misc_feature (30)..(33): n is a, c, g, or t

SEQ ID NO: 128 - DNA, 80 bp, Artificial Sequence; identical to SEQ ID NO: 22
  Synthetic
  misc_feature (3)..(34): n is a, c, g, or t

SEQ ID NO: 129 - DNA, 80 bp, Artificial Sequence; identical to SEQ ID NO: 23
  Synthetic
  misc_feature (30)..(36): n is a, c, g, or t

SEQ ID NO: 130 - DNA, 80 bp, Artificial Sequence; identical to SEQ ID NO: 24
  Synthetic
  misc_feature (27)..(34): n is a, c, g, or t

SEQ ID NO: 131 - DNA, 80 bp, Artificial Sequence; identical to SEQ ID NO: 25
  Synthetic
  misc_feature (28)..(36): n is a, c, g, or t

SEQ ID NO: 132 - DNA, 80 bp, Artificial Sequence; identical to SEQ ID NO: 26
  Synthetic
  misc_feature (28)..(31): n is a, c, g, or t

SEQ ID NO: 133 - DNA, 80 bp, Artificial Sequence; identical to SEQ ID NO: 27
  Synthetic
  misc_feature (27)..(31): n is a, c, g, or t

SEQ ID NO: 134 - DNA, 80 bp, Artificial Sequence; identical to SEQ ID NO: 28
  Synthetic
  misc_feature (29)..(36): n is a, c, g, or t

SEQ ID NO: 135 - DNA, 80 bp, Artificial Sequence; identical to SEQ ID NO: 29
  Synthetic
  misc_feature (31)..(34): n is a, c, g, or t

SEQ ID NO: 136 - DNA, 80 bp, Artificial Sequence; identical to SEQ ID NO: 30
  Synthetic
  misc_feature (28)..(32): n is a, c, g, or t

SEQ ID NO: 137 - DNA, 80 bp, Artificial Sequence; identical to SEQ ID NO: 31
  Synthetic
  misc_feature (26)..(31): n is a, c, g, or t

SEQ ID NO: 138 - DNA, 80 bp, Artificial Sequence; identical to SEQ ID NO: 32
  Synthetic
  misc_feature (22)..(38): n is a, c, g, or t

SEQ ID NO: 139 - DNA, 80 bp, Artificial Sequence; identical to SEQ ID NO: 33
  Synthetic
  misc_feature (29)..(35): n is a, c, g, or t

SEQ ID NO: 140 - DNA, 80 bp, Artificial Sequence; identical to SEQ ID NO: 34
  Synthetic
  misc_feature (29)..(31): n is a, c, g, or t

SEQ ID NO: 141 - DNA, 80 bp, Artificial Sequence; identical to SEQ ID NO: 35
  Synthetic
  misc_feature (22)..(36): n is a, c, g, or t

SEQ ID NO: 142 - DNA, 80 bp, Artificial Sequence; identical to SEQ ID NO: 36
  Synthetic
  misc_feature (24)..(35): n is a, c, g, or t

SEQ ID NO: 143 - DNA, 80 bp, Artificial Sequence; identical to SEQ ID NO: 37
  Synthetic
  misc_feature (20)..(36): n is a, c, g, or t

SEQ ID NO: 144 - DNA, 80 bp, Artificial Sequence; identical to SEQ ID NO: 38
  Synthetic
  misc_feature (6)..(34): n is a, c, g, or t

SEQ ID NO: 145 - DNA, 80 bp, Artificial Sequence; identical to SEQ ID NO: 39
  Synthetic
  misc_feature (24)..(34): n is a, c, g, or t

SEQ ID NO: 146 - DNA, 80 bp, Artificial Sequence; identical to SEQ ID NO: 40
  Synthetic
  misc_feature (32)..(38): n is a, c, g, or t

SEQ ID NO: 147 - DNA, 80 bp, Artificial Sequence; identical to SEQ ID NO: 41
  Synthetic
  misc_feature (23)..(38): n is a, c, g, or t

SEQ ID NO: 148 - DNA, 80 bp, Artificial Sequence; identical to SEQ ID NO: 42
  Synthetic
  misc_feature (20)..(32): n is a, c, g, or t

SEQ ID NO: 149 - DNA, 80 bp, Artificial Sequence; identical to SEQ ID NO: 43
  Synthetic
  misc_feature (27)..(33): n is a, c, g, or t

SEQ ID NO: 150 - DNA, 80 bp, Artificial Sequence
  Synthetic
  misc_feature (2)..(40): n is a, c, g, or t
cnnnnnnnnnnnnnnnnnnnnnnnnnnnnnnnnnnnnnnnctgtggatcctatagaagatttgcatcttttgtgtgatga

SEQ ID NO: 151 - DNA, 80 bp, Artificial Sequence; identical to SEQ ID NO: 45
  Synthetic
  misc_feature (30)..(32): n is a, c, g, or t

SEQ ID NO: 152 - DNA, 80 bp, Artificial Sequence; identical to SEQ ID NO: 46
  Synthetic
  misc_feature (25)..(38): n is a, c, g, or t

SEQ ID NO: 153 - DNA, 80 bp, Artificial Sequence; identical to SEQ ID NO: 47
  Synthetic
  misc_feature (31)..(36): n is a, c, g, or t

SEQ ID NO: 154 - DNA, 80 bp, Artificial Sequence; identical to SEQ ID NO: 48
  Synthetic
  misc_feature (1)..(39): n is a, c, g, or t

SEQ ID NO: 155 - DNA, 80 bp, Artificial Sequence; identical to SEQ ID NO: 49
  Synthetic
  misc_feature (36)..(41): n is a, c, g, or t

SEQ ID NO: 156 - DNA, 80 bp, Artificial Sequence; identical to SEQ ID NO: 50
  Synthetic
  misc_feature (24)..(36): n is a, c, g, or t

SEQ ID NO: 157 - DNA, 80 bp, Artificial Sequence; identical to SEQ ID NO: 51
  Synthetic
  misc_feature (31)..(35): n is a, c, g, or t

SEQ ID NO: 158 - DNA, 80 bp, Artificial Sequence; identical to SEQ ID NO: 52
  Synthetic
  misc_feature (26)..(34): n is a, c, g, or t

SEQ ID NO: 159 - DNA, 80 bp, Artificial Sequence; identical to SEQ ID NO: 53
  Synthetic
  misc_feature (29)..(37): n is a, c, g, or t

SEQ ID NO: 160 - DNA, 80 bp, Artificial Sequence; identical to SEQ ID NO: 54
  Synthetic
  misc_feature (5)..(37): n is a, c, g, or t

SEQ ID NO: 161 - DNA, 80 bp, Artificial Sequence; identical to SEQ ID NO: 55
  Synthetic
  misc_feature (24)..(42): n is a, c, g, or t

SEQ ID NO: 162 - DNA, 80 bp, Artificial Sequence; identical to SEQ ID NO: 56
  Synthetic
  misc_feature (21)..(33): n is a, c, g, or t

SEQ ID NO: 163 - DNA, 80 bp, Artificial Sequence; identical to SEQ ID NO: 57
  Synthetic
  misc_feature (4)..(33): n is a, c, g, or t

SEQ ID NO: 164 - DNA, 80 bp, Artificial Sequence; identical to SEQ ID NO: 58
  Synthetic
  misc_feature (23)..(33): n is a, c, g, or t

SEQ ID NO: 165 - DNA, 80 bp, Artificial Sequence
  Synthetic
  misc_feature (30)..(30): n is a, c, g, or t
ctggatgattagttcagagatatatcaaanttccccctccctgtggatcctatagaggatttgcatcttttgtgtgatca

SEQ ID NO: 166 - DNA, 80 bp, Artificial Sequence
  Synthetic
  misc_feature (37)..(37): n is a, c, g, or t
ctggatgattagttcagagatatatcaaaattccccntccctgtggatcctatagaggatttgcatcttttgtgtgatca

SEQ ID NO: 167 - DNA, 80 bp, Artificial Sequence
  Synthetic
  misc_feature (31)..(33): n is a, c, g, or t
ctggatgattagttcagagatatatcaaaannncccctccctgtggatcctatagaggatttgcatcttttgtgtgatca

SEQ ID NO: 168 - DNA, 80 bp, Artificial Sequence
  Synthetic
atcgatcattagttcagagatatatcaaaattccccctccctgtggatcctatagaggatttgcatcttttgtgtgatca

SEQ ID NO: 169 - DNA, 80 bp, Artificial Sequence
  Synthetic
  misc_feature (37)..(37): n is a, c, g, or t
atggatgattagttcagagatatatcaaaattccccntccctgtggatcctatagaggatttgcatcttttgtgtgatca

SEQ ID NO: 170 - DNA, 80 bp, Artificial Sequence
  Synthetic
  misc_feature (37)..(37): n is a, c, g, or t
ctcgatgattagttcagagatatatcaaaattccccntccctgtggatcctatagaggatttgcatcttttgtgtgatca

SEQ ID NO: 171 - DNA, 80 bp, Artificial Sequence; identical to SEQ ID NO: 110
  Synthetic

SEQ ID NO: 172 - DNA, 80 bp, Artificial Sequence
  Synthetic
ctctatgattagttcagagatatatcaaaattccccctccctgtggatcctatagaggatttgcatcttttgtgtgatca

SEQ ID NO: 173 - DNA, 80 bp, Artificial Sequence
  Synthetic
  misc_feature (4)..(43): n is a, c, g, or t
ctgnnnnnnnnnnnnnnnnnnnnnnnnnnnnnnnnnnnnnnnntggatcctatagaggatttgcatcttttgtgtgatca

SEQ ID NO: 174 - DNA, 80 bp, Artificial Sequence; identical to SEQ ID NO: 108
  Synthetic

SEQ ID NO: 175 - DNA, 80 bp, Artificial Sequence
  Synthetic
  misc_feature (25)..(38): n is a, c, g, or t
ctggatgattagttcagagatatannnnnnnnnnnnnnccctgtggatcctatagaggatttgcatcttttgtgtgatca

SEQ ID NO: 176 - DNA, 80 bp, Artificial Sequence
  Synthetic
ctggatgattatttcagagatatatcaaaattccccctccctgtggatcctatagaggatttgcatcttttgtgtgatca

SEQ ID NO: 177 - DNA, 80 bp, Artificial Sequence; identical to SEQ ID NO: 113
  Synthetic

SEQ ID NO: 178 - DNA, 80 bp, Artificial Sequence; identical to SEQ ID NO: 109
  Synthetic

SEQ ID NO: 179 - DNA, 80 bp, Artificial Sequence; identical to SEQ ID NO: 59
  Synthetic

SEQ ID NO: 180 - DNA, 80 bp, Artificial Sequence; identical to SEQ ID NO: 60
  Synthetic

SEQ ID NO: 181 - DNA, 80 bp, Artificial Sequence; identical to SEQ ID NO: 9
  Synthetic

SEQ ID NO: 182 - DNA, 80 bp, Artificial Sequence; identical to SEQ ID NO: 62
  Synthetic

SEQ ID NO: 183 - DNA, 80 bp, Artificial Sequence; identical to SEQ ID NO: 63
  Synthetic

SEQ ID NO: 184 - DNA, 80 bp, Artificial Sequence; identical to SEQ ID NO: 64
  Synthetic

SEQ ID NO: 185 - DNA, 80 bp, Artificial Sequence; identical to SEQ ID NO: 65
  Synthetic

SEQ ID NO: 186 - DNA, 80 bp, Artificial Sequence; identical to SEQ ID NO: 66
  Synthetic

SEQ ID NO: 187 - DNA, 80 bp, Artificial Sequence; identical to SEQ ID NO: 67
  Synthetic

SEQ ID NO: 188 - DNA, 80 bp, Artificial Sequence; identical to SEQ ID NO: 68
  Synthetic

SEQ ID NO: 189 - DNA, 80 bp, Artificial Sequence; identical to SEQ ID NO: 69
  Synthetic

SEQ ID NO: 190 - DNA, 80 bp, Artificial Sequence; identical to SEQ ID NO: 70
  Synthetic

SEQ ID NO: 191 - DNA, 80 bp, Artificial Sequence; identical to SEQ ID NO: 71
  Synthetic

SEQ ID NO: 192 - DNA, 80 bp, Artificial Sequence; identical to SEQ ID NO: 72
  Synthetic

SEQ ID NO: 193 - DNA, 80 bp, Artificial Sequence; identical to SEQ ID NO: 73
  Synthetic

SEQ ID NO: 194 - DNA, 80 bp, Artificial Sequence; identical to SEQ ID NO: 74
  Synthetic

SEQ ID NO: 195 - DNA, 80 bp, Artificial Sequence; identical to SEQ ID NO: 75
  Synthetic

SEQ ID NO: 196 - DNA, 80 bp, Artificial Sequence; identical to SEQ ID NO: 76
  Synthetic

SEQ ID NO: 197 - DNA, 80 bp, Artificial Sequence; identical to SEQ ID NO: 77
  Synthetic

SEQ ID NO: 198 - DNA, 80 bp, Artificial Sequence; identical to SEQ ID NO: 78
  Synthetic

SEQ ID NO: 199 - DNA, 80 bp, Artificial Sequence; identical to SEQ ID NO: 79
  Synthetic

SEQ ID NO: 200 - DNA, 80 bp, Artificial Sequence; identical to SEQ ID NO: 80
  Synthetic

SEQ ID NO: 201 - DNA, 80 bp, Artificial Sequence; identical to SEQ ID NO: 81
  Synthetic

SEQ ID NO: 202 - DNA, 80 bp, Artificial Sequence; identical to SEQ ID NO: 82
  Synthetic

SEQ ID NO: 203 - DNA, 80 bp, Artificial Sequence; identical to SEQ ID NO: 83
  Synthetic

SEQ ID NO: 204 - DNA, 80 bp, Artificial Sequence; identical to SEQ ID NO: 84
  Synthetic

SEQ ID NO: 205 - DNA, 80 bp, Artificial Sequence; identical to SEQ ID NO: 85
  Synthetic

SEQ ID NO: 206 - DNA, 80 bp, Artificial Sequence; identical to SEQ ID NO: 86
  Synthetic

SEQ ID NO: 207 - DNA, 80 bp, Artificial Sequence; identical to SEQ ID NO: 87
  Synthetic

SEQ ID NO: 208 - DNA, 80 bp, Artificial Sequence; identical to SEQ ID NO: 88
  Synthetic

SEQ ID NO: 209 - DNA, 80 bp, Artificial Sequence; identical to SEQ ID NO: 89
  Synthetic
  misc_feature (28)..(37): n is a, c, g, or t

SEQ ID NO: 210 - DNA, 80 bp, Artificial Sequence; identical to SEQ ID NO: 90
  Synthetic

SEQ ID NO: 211 - DNA, 80 bp, Artificial Sequence; identical to SEQ ID NO: 91
  Synthetic

SEQ ID NO: 212 - DNA, 80 bp, Artificial Sequence; identical to SEQ ID NO: 108
  Synthetic

SEQ ID NO: 213 - DNA, 80 bp, Artificial Sequence
  Synthetic
ctggattattagttcagagatatatcaaaattccccctccctgtggatcctatagaggatttgcatcttttgtgtgatca

SEQ ID NO: 214 - DNA, 80 bp, Artificial Sequence; identical to SEQ ID NO: 110
  Synthetic

SEQ ID NO: 215 - DNA, 80 bp, Artificial Sequence
  Synthetic
  misc_feature (15)..(15): n is a, c, g, or t
ctggatgattagttnagagatatatcaaaattccccctccctgtggatcctatagaggatttgcatcttttgtgtgatca

SEQ ID NO: 216 - DNA, 80 bp, Artificial Sequence
  Synthetic
  misc_feature (25)..(33): n is a, c, g, or t
ctggatgattagttcagagatatannnnnnnnncccccccctgtggatcctatagaggatttgcatcttttgtgtgatca

SEQ ID NO: 217 - DNA, 80 bp, Artificial Sequence
  Synthetic
ctggatgattagttcagagatatatcaaaattcctcctccctgtggatcctatagaggatttgcatcttttgtgtgatca

SEQ ID NO: 218 - DNA, 80 bp, Artificial Sequence
  Synthetic
ctggatgattagttcagagatatatgaaaattccccctccctgtggatcctatagaggatttgcatcttttgtgtgatca

SEQ ID NO: 219 - DNA, 80 bp, Artificial Sequence
  Synthetic
ctggatgattagttcagagatgtgtcggggtgccccctccctgtggatcctatagaggatttgcatcttttgtgtgatca

SEQ ID NO: 220 - DNA, 80 bp, Artificial Sequence; identical to SEQ ID NO: 176
  Synthetic

SEQ ID NO: 221 - DNA, 80 bp, Artificial Sequence; identical to SEQ ID NO: 113
  Synthetic

SEQ ID NO: 222 - DNA, 80 bp, Artificial Sequence
  Synthetic
ctgtatgataagttcagagatatatcaaaattccccctccctgtggatcctatagaggatttgcatcttttgtgtgatca

SEQ ID NO: 223 - DNA, 80 bp, Artificial Sequence; identical to SEQ ID NO: 109
  Synthetic

SEQ ID NO: 224 - DNA, 80 bp, Artificial Sequence
  Synthetic
cttcaagataagttcagagatatatcaaaattccccctccctgtggatcctatagaggatttgcatcttttgtgtgatca

SEQ ID NO: 225 - DNA, 80 bp, Artificial Sequence
  Synthetic
ctttatgattagttcagagatatatcaaaattccccctccctgtggatcctatagaggatttgcatcttttgtgtgatca

SEQ ID NO: 226 - DNA, 80 bp, Artificial Sequence; identical to SEQ ID NO: 60
  Synthetic

SEQ ID NO: 227 - DNA, 80 bp, Artificial Sequence; identical to SEQ ID NO: 59
  Synthetic

SEQ ID NO: 228 - DNA, 80 bp, Artificial Sequence; identical to SEQ ID NO: 62
  Synthetic

SEQ ID NO: 229 - DNA, 80 bp, Artificial Sequence; identical to SEQ ID NO: 63
  Synthetic

SEQ ID NO: 230 - DNA, 80 bp, Artificial Sequence; identical to SEQ ID NO: 64
  Synthetic

SEQ ID NO: 231 - DNA, 80 bp, Artificial Sequence; identical to SEQ ID NO: 67
  Synthetic

SEQ ID NO: 232 - DNA, 80 bp, Artificial Sequence; identical to SEQ ID NO: 76
  Synthetic

SEQ ID NO: 233 - DNA, 80 bp, Artificial Sequence; identical to SEQ ID NO: 99
  Synthetic

SEQ ID NO: 234 - DNA, 80 bp, Artificial Sequence; identical to SEQ ID NO: 70
  Synthetic

SEQ ID NO: 235 - DNA, 80 bp, Artificial Sequence; identical to SEQ ID NO: 100
  Synthetic

SEQ ID NO: 236 - DNA, 80 bp, Artificial Sequence
  Synthetic
ctggatgattagttcagagacacattggggtgccccctccctgtggatcctatagaagatttgcatcttttgtgtgatga

SEQ ID NO: 237 - DNA, 80 bp, Artificial Sequence; identical to SEQ ID NO: 85
  Synthetic

SEQ ID NO: 238 - DNA, 80 bp, Artificial Sequence; identical to SEQ ID NO: 68
  Synthetic

SEQ ID NO: 239 - DNA, 80 bp, Artificial Sequence; identical to SEQ ID NO: 72
  Synthetic

SEQ ID NO: 240 - DNA, 80 bp, Artificial Sequence; identical to SEQ ID NO: 75
  Synthetic

SEQ ID NO: 241 - DNA, 80 bp, Artificial Sequence
  Synthetic
ctggatgattagttcagacatatattggggtgccccctccctgtggatcctatagaagatttgcatcttttgtgtgatga

SEQ ID NO: 242 - DNA, 80 bp, Artificial Sequence
  Synthetic
ctggatgattagttcagagatacattaaaatgccccctccctgtggatcctatagaagatttgcatcttttgtgtgatga

SEQ ID NO: 243 - DNA, 80 bp, Artificial Sequence
  Synthetic
ctggatgattagttcagagatatgttgaaatgccccctccctgtggatcctatagaagatttgcatcttttgtgtgatga

SEQ ID NO: 244 - DNA, 80 bp, Artificial Sequence; identical to SEQ ID NO: 88
  Synthetic

SEQ ID NO: 245 - DNA, 80 bp, Artificial Sequence; identical to SEQ ID NO: 101
  Synthetic

SEQ ID NO: 246 - DNA, 80 bp, Artificial Sequence; identical to SEQ ID NO: 91
  Synthetic

SEQ ID NO: 247 - DNA, 80 bp, Artificial Sequence; identical to SEQ ID NO: 73
  Synthetic

SEQ ID NO: 248 - DNA, 80 bp, Artificial Sequence; identical to SEQ ID NO: 78
  Synthetic

SEQ ID NO: 249 - DNA, 80 bp, Artificial Sequence; identical to SEQ ID NO: 109
  Synthetic

SEQ ID NO: 250 - DNA, 80 bp, Artificial Sequence; identical to SEQ ID NO: 110
  Synthetic

SEQ ID NO: 251 - DNA, 80 bp, Artificial Sequence
  Synthetic
atttatgattagttcagagatatatcaaaattccccctccctgtggatcctatagaggatttgcatcttttgtgtgatca

SEQ ID NO: 252 - DNA, 80 bp, Artificial Sequence
  Synthetic
cacgatgattagttcagagatatatcaaaattccccctccctgtggatcctatagaggatttgcatcttttgtgtgatca

SEQ ID NO: 253 - DNA, 80 bp, Artificial Sequence
  Synthetic
ctccatgattagttcagagatatatcaaaattccccctccctgtggatcctatagaggatttgcatcttttgtgtgatca

SEQ ID NO: 254 - DNA, 80 bp, Artificial Sequence; identical to SEQ ID NO: 172
  Synthetic

SEQ ID NO: 255 - DNA, 80 bp, Artificial Sequence
  Synthetic
ctggatgataagttcagagatatatcaaaattccccctccctgtggatcctatagaggatttgcatcttttgtgtgatca

SEQ ID NO: 256 - DNA, 80 bp, Artificial Sequence
  Synthetic
ctggatgattagttcagagatatattaaaattccccctccctgtggatcctatagaggatttgcatcttttgtgtgatca

SEQ ID NO: 257 - DNA, 80 bp, Artificial Sequence; identical to SEQ ID NO: 176
  Synthetic

SEQ ID NO: 258 - DNA, 80 bp, Artificial Sequence; identical to SEQ ID NO: 113
  Synthetic

SEQ ID NO: 259 - DNA, 80 bp, Artificial Sequence
  Synthetic
cttgatgattagttcagagatatatcaaaattccccctccctgtggatcctatagaggatttgcatcttttgtgtgatca

SEQ ID NO: 260 - DNA, 80 bp, Artificial Sequence; identical to SEQ ID NO: 60
  Synthetic

SEQ ID NO: 261 - DNA, 80 bp, Artificial Sequence; identical to SEQ ID NO: 9
  Synthetic

SEQ ID NO: 262 - DNA, 80 bp, Artificial Sequence; identical to SEQ ID NO: 59
  Synthetic

SEQ ID NO: 263 - DNA, 80 bp, Artificial Sequence; identical to SEQ ID NO: 62
  Synthetic

SEQ ID NO: 264 - DNA, 80 bp, Artificial Sequence; identical to SEQ ID NO: 63
  Synthetic

SEQ ID NO: 265 - DNA, 80 bp, Artificial Sequence; identical to SEQ ID NO: 64
  Synthetic

SEQ ID NO: 266 - DNA, 80 bp, Artificial Sequence; identical to SEQ ID NO: 67
  Synthetic

SEQ ID NO: 267 - DNA, 80 bp, Artificial Sequence; identical to SEQ ID NO: 68
  Synthetic

SEQ ID NO: 268 - DNA, 80 bp, Artificial Sequence; identical to SEQ ID NO: 70
  Synthetic

SEQ ID NO: 269 - DNA, 80 bp, Artificial Sequence; identical to SEQ ID NO: 65
  Synthetic

SEQ ID NO: 270 - DNA, 80 bp, Artificial Sequence; identical to SEQ ID NO: 71
  Synthetic

SEQ ID NO: 271 - DNA, 80 bp, Artificial Sequence; identical to SEQ ID NO: 66
  Synthetic

SEQ ID NO: 272 - DNA, 80 bp, Artificial Sequence; identical to SEQ ID NO: 236
  Synthetic

SEQ ID NO: 273 - DNA, 80 bp, Artificial Sequence; identical to SEQ ID NO: 75
  Synthetic

SEQ ID NO: 274 - DNA, 80 bp, Artificial Sequence; identical to SEQ ID NO: 69
  Synthetic

SEQ ID NO: 275 - DNA, 80 bp, Artificial Sequence; identical to SEQ ID NO: 72
  Synthetic

SEQ ID NO: 276 - DNA, 80 bp, Artificial Sequence
  Synthetic
ctggatgattagttcagagatatgttggaatgccccctccctgtggatcctatagaagatttgcatcttttgtgtgatga

SEQ ID NO: 277 - DNA, 80 bp, Artificial Sequence
  Synthetic
ctggatgattagttcagagatatgttgaggtgccccctccctgtggatcctatagaagatttgcatcttttgtgtgatga

SEQ ID NO: 278 - DNA, 80 bp, Artificial Sequence; identical to SEQ ID NO: 86
  Synthetic

SEQ ID NO: 279 - DNA, 80 bp, Artificial Sequence; identical to SEQ ID NO: 76
  Synthetic

SEQ ID NO: 280 - DNA, 80 bp, Artificial Sequence; identical to SEQ ID NO: 82
  Synthetic

SEQ ID NO: 281 - DNA, 80 bp, Artificial Sequence
  Synthetic
ctggatgattagttcagagacacattgggatgccccctccctgtggatcctatagaagatttgcatcttttgtgtgatga

SEQ ID NO: 282 - DNA, 80 bp, Artificial Sequence; identical to SEQ ID NO: 243
  Synthetic

SEQ ID NO: 283 - DNA, 80 bp, Artificial Sequence; identical to SEQ ID NO: 84
  Synthetic

SEQ ID NO: 284 - DNA, 80 bp, Artificial Sequence
  Synthetic
ctggatgattagttcagagatatattgaagtgccccctccctgtggatcctatagaagatttgcatcttttgtgtgatga

SEQ ID NO: 285 - DNA, 80 bp, Artificial Sequence; identical to SEQ ID NO: 73
  Synthetic

SEQ ID NO: 286 - DNA, 80 bp, Artificial Sequence; identical to SEQ ID NO: 85
  Synthetic

SEQ ID NO: 287 - DNA, 80 bp, Artificial Sequence; identical to SEQ ID NO: 83
  Synthetic

SEQ ID NO: 288 - DNA, 80 bp, Artificial Sequence; identical to SEQ ID NO: 77
  Synthetic

SEQ ID NO: 289 - DNA, 80 bp, Artificial Sequence; identical to SEQ ID NO: 99
  Synthetic

SEQ ID NO: 290 - DNA, 80 bp, Artificial Sequence; identical to SEQ ID NO: 100
  Synthetic

SEQ ID NO: 291 - DNA, 80 bp, Artificial Sequence; identical to SEQ ID NO: 87
  Synthetic

SEQ ID NO: 292 - DNA, 80 bp, Artificial Sequence; identical to SEQ ID NO: 91
  Synthetic

SEQ ID NO: 293 - DNA, 80 bp, Artificial Sequence
  Synthetic
ctggatgattagttcagagatgtattgaagtgccccctccctgtggatcctatagaagatttgcatcttttgtgtgatga

SEQ ID NO: 294 - DNA, 80 bp, Artificial Sequence
  Synthetic
ctggatgattagttcagagatgtgttgggatgccccctccctgtggatcctatagaagatttgcatcttttgtgtgatga

SEQ ID NO: 295 - DNA, 80 bp, Artificial Sequence; identical to SEQ ID NO: 109
  Synthetic

SEQ ID NO: 296 - DNA, 80 bp, Artificial Sequence; identical to SEQ ID NO: 110
  Synthetic

SEQ ID NO: 297 - DNA, 80 bp, Artificial Sequence; identical to SEQ ID NO: 108
  Synthetic

SEQ ID NO: 298 - DNA, 80 bp, Artificial Sequence; identical to SEQ ID NO: 213
  Synthetic

SEQ ID NO: 299 - DNA, 80 bp, Artificial Sequence
  Synthetic
ctggatgattagttcagagacatatcaaaattccccctccctgtggatcctatagaggatttgcatcttttgtgtgatca

SEQ ID NO: 300 - DNA, 80 bp, Artificial Sequence
  Synthetic
gtggatgattagttcagagatatatcaaaattccccctccctgtggatcctatagaggatttgcatcttttgtgtgatca

SEQ ID NO: 301 - DNA, 80 bp, Artificial Sequence
  Synthetic
ctgcatgattacttcagagatatatcaaaattccccctccctgtggatcctatagaggatttgcatcttttgtgtgatca

SEQ ID NO: 302 - DNA, 80 bp, Artificial Sequence
  Synthetic
ctgcatgattatttcagagatatatcaaaattccccctccctgtggatcctatagaggatttgcatcttttgtgtgatca

SEQ ID NO: 303 - DNA, 80 bp, Artificial Sequence; identical to SEQ ID NO: 225
  Synthetic

SEQ ID NO: 304 - DNA, 80 bp, Artificial Sequence
  Synthetic
ctgtattattagttcagagatatatcaaaattccccctccctgtggatcctatagaggatttgcatcttttgtgtgatca

SEQ ID NO: 305 - DNA, 80 bp, Artificial Sequence
  Synthetic
ctgtatgattagttcagagatatgttagggtgccccctccctgtggatcctatagaggatttgcatcttttgtgtgatca

SEQ ID NO: 306 - DNA, 80 bp, Artificial Sequence
  Synthetic
caggatgattagttcagagatatatcaaaattccccctccctgtggatcctatagaggatttgcatcttttgtgtgatca

SEQ ID NO: 307 - DNA, 80 bp, Artificial Sequence; identical to SEQ ID NO: 222
  Synthetic

SEQ ID NO: 308 - DNA, 80 bp, Artificial Sequence
  Synthetic
catgatgattagttcagagatatatcaaaattccccctccctgtggatcctatagaggatttgcatcttttgtgtgatca

SEQ ID NO: 309 - DNA, 80 bp, Artificial Sequence
  Synthetic
ctggatgattagttcggagatatatcaaaattccccctccctgtggatcctatagaggatttgcatcttttgtgtgatca

SEQ ID NO: 310 - DNA, 80 bp, Artificial Sequence
  Synthetic
ctggatgattagttcagagatatgttggggtgccccctccctgtggatcctatagaggatttgcatcttttgtgtgatca

SEQ ID NO: 311 - DNA, 80 bp, Artificial Sequence
  Synthetic
ctggatgattagttcagagatatattggggtgccccctccctgtggatcctatagaggatttgcatcttttgtgtgatca

SEQ ID NO: 312 - DNA, 80 bp, Artificial Sequence
  Synthetic
ctggatgattagttcagagatatatcaagattccccctccctgtggatcctatagaggatttgcatcttttgtgtgatca

SEQ ID NO: 313 - DNA, 80 bp, Artificial Sequence
  Synthetic
ctggatgattagttcagagatatatcaaaattgcccctccctgtggatcctatagaggatttgcatcttttgtgtgatca

SEQ ID NO: 314 - DNA, 80 bp, Artificial Sequence
  Synthetic
ctggatgattagttcagagatatatcaaaattccccctcgctgtggatcctatagaggatttgcatcttttgtgtgatca

SEQ ID NO: 315 - DNA, 80 bp, Artificial Sequence
  Synthetic
atgtattattagttgagagatatatcaaaattccccctccctgtggatcctatagaggatttgcatcttttgtgtgatca

SEQ ID NO: 316 - DNA, 80 bp, Artificial Sequence
  Synthetic
ctggatgattagttcagagagatatcaaaattccccctccctgtggatcctatagaggatttgcatcttttgtgtgatca

SEQ ID NO: 317 - DNA, 80 bp, Artificial Sequence
  Synthetic
ctggatgattagttcagagacatattggggtgccccctccctgtggatcctatagaggatttgcatcttttgtgtgatca

SEQ ID NO: 318 - DNA, 80 bp, Artificial Sequence
  Synthetic
ctcgatgattagttcagatatatatcaaaattccccctccctgtggatcctatagaggatttgcatcttttgtgtgatca

SEQ ID NO: 319 - DNA, 80 bp, Artificial Sequence
  Synthetic
ctggatgattagttcagagacacatcaaaattccccctccctgtggatcctatagaggatttgcatcttttgtgtgatca

SEQ ID NO: 320 - DNA, 80 bp, Artificial Sequence
  Synthetic
atgtatgattagttcagagatatatcaaaattccccctccctgtggatcctatagaggatttgcatcttttgtgtgatca

SEQ ID NO: 321 - DNA, 34 bp, Artificial Sequence
  Synthetic
ttagttcagagatatattaaaatgccccctccct

SEQ ID NO: 322 - DNA, 34 bp, Artificial Sequence
  Synthetic
  misc_feature (16)..(23): n is a, c, g, or t
ttagttcagagatatnnnnnnnngccccctccct

SEQ ID NO: 323 - DNA, 34 bp, Artificial Sequence
  Synthetic
  misc_feature (17)..(28): n is a, c, g, or t
ttagttcagagatatannnnnnnnnnnnctccct